(19) United States
(12) Patent Application Publication
(10) Pub. No.: US 2012/0122104 A1
(43) Pub. Date: May 17, 2012

(54) Triple-Stranded Nucleobase Structures and Uses Thereof

(75) Inventors: Neil Straus, Emeryville, CA (US); Stefan Matysiak, Somerville, MA (US); Eric Anderson, Redwood City, CA (US)

(73) Assignee: Applera Corporation, Foster City, CA (US)

(21) Appl. No.: 11/690,656

(22) Filed: Mar. 23, 2007

Related U.S. Application Data
(60) Provisional application No. 60/786,155, filed on Mar. 26, 2006.

(51) Int. Cl. C12Q 1/68; G01N 27/447; G01N 21/64

(52) U.S. Cl. 435/6.12; 204/450

(57) ABSTRACT

The present disclosure relates to compositions and methods of using triplex structures generated by a duplex of a polypurine tract and complementary polypyrimidine tract and a triplex-forming nucleobase polymer that hydrogen bonds to both the purine and pyrimidine bases of the polypurine-polypyrimidine duplex.

### 1. CROSS REFERENCE TO RELATED APPLICATION

This application claims the benefit under 35 U.S.C. §119(e) of U.S. provisional application No. 60/786,155 filed Mar. 26, 2006, the disclosure of which is incorporated herein by reference in its entirety.

### 2. INTRODUCTION

Nucleic acids exist as various secondary structures, such as single-stranded, double-stranded, and triple-stranded forms. Multi-stranded nucleic acids arise from the interplay of a number of molecular forces that include hydrogen bonding, electrostatic interactions (e.g., charge-charge, charge-dipole, and dipole-dipole), van der Waals interactions, and hydrophobic (entropic) effects. One factor stabilizing formation of multi-stranded nucleic acids is base stacking, which occurs from a combination of van der Waals interactions, dipole-dipole interactions, and hydrophobic effect. Hydrogen bonding is another stabilizing factor and provides the specificity required for the complementarity of bases, such as the complementarity of bases in deoxyribonucleic acid (DNA) essential for DNA replication and transcription. Substantial non-complementary of the bases between two nucleic acid strands prevents proper base-stacking required for forming stable multi-stranded structures.

Triple-stranded nucleic acids are known to form between a Watson-Crick base-paired double-stranded nucleic acid and a third nucleobase polymer strand, sometimes referred to as “triplex forming oligonucleotides” or “TFO.” At least two motifs or types of triplexes are known. In one motif, a polypyrimidine binds parallel to the purine strand of a Watson Crick base-paired polypyrimidine:polypurine duplex to form C:G*C⁺ and T:A*T triplexes (C⁺=protonated cytosine) via Hoogsteen hydrogen bonding. In a second motif, a G-rich polypurine binds antiparallel to the purine strand of a Watson-Crick base-paired polypyrimidine:polypurine duplex to form C:G*G and T:A*T or C:G*G and T:A*A triplexes via reverse Hoogsteen hydrogen bonding (see, e.g., Roberts and Crothers, 1992, Science 258:1463-1466). In either case, the third strand forms hydrogen bonds with only the polypurine strand of the Watson-Crick base-paired duplex substrate. Exemplary triplexes of the pyrimidine motif form when a double-stranded polyU:polyA interacts with a third strand of polyU to form a triple-stranded structure of polyU:polyA*polyU while double-stranded polyG:polyC interacting with a third strand of polyC forms a triple-stranded structure of polyC:polyG*polyC (see, e.g., Felsenfeld et al., 1957, J Am Chem Soc 79:2023). Triplexes formed of the purine motif require high divalent cation concentrations and can be destabilized by formation of intramolecular and intermolecular interactions of the G-rich purine strand.

Peptide nucleic acids (PNAs) having a backbone of N-(2-aminoethyl)glycine units and a sequence of polypyrimidines can also interact with complementary polypurine tracts of DNAs to form triplexes of PNA:DNA*PNA (Nielsen et al., 1994, J Mol Recog 7:164-170; Cherny et al., 1993, Proc. Natl. Acad. Sci. USA 90:1667-1670). In these structures, a polypyrimidine tract on one PNA strand is Watson-Crick base-paired with the complementary polypurine tract in the DNA while a polypyrimidine tract on the second PNA strand is Hoogsteen base-paired with the polypurine tract of the DNA. Where the substrate is a double-stranded DNA containing a polypurine tract, triplex structures form by strand invasion of the double-stranded nucleic acid by a PNA strand with a complementary polypyrimidine tract to form a Watson-Crick based paired PNA:DNA duplex. The second PNA strand is Hoogsteen base-paired with the polypurine on the DNA strand to form the third strand of the triplex structure while the second DNA strand is displaced by the strand invasion reaction to generate a “P-loop” (Bukanov et al., 1998, Proc Natl Acad Sci USA 95:5516-5520; Lesnik et al., 1997, Nucleic Acids Res. 25:568-574; and WO 99/55914). Aside from the displaced DNA strand, the triple-stranded structures formed by PNAs and duplex DNA are similar to the triplex structures of pyrimidine motif described above. In the present disclosure, alternative triplex structures are described that exploit hydrogen bonding information available in the major groove of a Watson-Crick paired polynucleotide duplex.

### 3. BRIEF DESCRIPTION OF THE FIGURES

FIG. 1 illustrates the Watson-Crick hydrogen bonding scheme of bases in B-form duplex DNA.

FIG. 2A illustrates Hoogsteen hydrogen bonding scheme between a purine base and a pyrimidine base. FIG. 2B illustrates Reverse Hoogsteen hydrogen bonding scheme between a purine base and a pyrimidine base, where the bases are in the anti configuration.

FIG. 3A illustrates triplex structures in which a pyrimidine base on a third strand hydrogen bonds to a purine base of a Watson-Crick base-paired duplex via Hoogsteen hydrogen bonding. FIG. 3B illustrates triplex structures in which a pyrimidine base on a third strand hydrogen bonds to a purine base of a Watson-Crick base-paired duplex via Reverse Hoogsteen hydrogen bonding.

FIG. 4A illustrates a Straus-Matysiak hydrogen bonding pattern between a purine base of a triplex forming nucleobase polymer (TFNP) and a purine:pyrimidine base-pair of a Watson-Crick base-paired duplex. The purine base of the third strand forms hydrogen bonds with both bases of the purine:pyrimidine base-pair. FIG. 4B illustrates a Reverse Straus-Matysiak hydrogen bonding scheme between a iG purine base (iG=isoguanine) of a TFNP and a C:G base-pair.

FIG. 5 illustrates a Straus-Matysiak and Reverse Straus-Matysiak hydrogen bonding schemes between purine base 2,6-diaminoadenine (a) of a TFNP and a Watson-Crick base-paired A:T base-pair of a duplex.

FIG. 6A is a ball and stick model of a triplex formed between a duplex DNA and a TFNP, where the TFNP is a PNA with an N-(2-aminoethyl)-β-alanine backbone (see, Nielsen et al., 1997, “Peptide nucleic acid (PNA). A DNA mimic with a pseudopeptide backbone,” Chemical Society Reviews pp. 73-78). The third strand winds along the major groove in anti-parallel orientation from the bottom to the top. FIGS. 6B-6D represent various perspectives of a space filling model of a PNA with an N-(2-aminoethyl)-β-alanine backbone, where the third strand is shown in both anti-parallel and parallel orientations. FIG. 6B shows both orientations of the TFNP strand, with the bottom portion being carboxyl to amino orientation and top portion being amino to carboxyl orientation; the change over occurs in the center of the model. FIG. 6C highlights the lower half of the model where the PNA third strand is in a parallel configuration while FIG. 6D highlights the upper half of the model where the PNA third strand is in an anti-parallel configuration.

### 4. DETAILED DESCRIPTION

It is to be understood that both the foregoing general description, including the drawings, and the following detailed description are exemplary and explanatory only and are not restrictive of this disclosure. In this disclosure, the use of the singular includes the plural (and vice versa) unless specifically stated otherwise. Also, the use of “or” means “and/or” unless stated otherwise. Similarly, “comprise,” “comprises,” “comprising” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of”

The section headings used herein are for organizational purposes only and not to be construed as limiting the subject matter described.

4.1 Definitions

As used herein, the following terms are intended to have the following meanings:

“Alkyl” by itself or as part of another substituent refers to a saturated or unsaturated branched, straight-chain or cyclic monovalent hydrocarbon radical having the stated number of carbon atoms (i.e., C1-C6 means one to six carbon atoms) that is derived by the removal of one hydrogen atom from a single carbon atom of a parent alkane, alkene or alkyne. Alkyl groups can include, but are not limited to, methyl; ethyls such as ethanyl, ethenyl, ethynyl; propyls such as propan-1-yl, propan-2-yl, cyclopropan-1-yl, prop-1-en-1-yl, prop-1-en-2-yl, prop-2-en-1-yl, cycloprop-1-en-1-yl; cycloprop-2-en-1-yl, prop-1-yn-1-yl, prop-2-yn-1-yl, etc.; butyls such as butan-1-yl, butan-2-yl, 2-methyl-propan-1-yl, 2-methyl-propan-2-yl, cyclobutan-1-yl, but-1-en-1-yl, but-1-en-2-yl, 2-methyl-prop-1-en-1-yl, but-2-en-1-yl, but-2-en-2-yl, buta-1,3-dien-1-yl, buta-1,3-dien-2-yl, cyclobut-1-en-1-yl, cyclobut-1-en-3-yl, cyclobuta-1,3-dien-1-yl, but-1-yn-1-yl, but-1-yn-3-yl, but-3-yn-1-yl, etc.; and the like. The term “alkyl” is specifically intended to include groups having any degree or level of saturation, i.e., groups having exclusively single carbon-carbon bonds, groups having one or more double carbon-carbon bonds, groups having one or more triple carbon-carbon bonds and groups having mixtures of single, double and triple carbon-carbon bonds. Where a specific level of saturation is intended, the expressions “alkanyl,” “alkenyl,” and “alkynyl” are used. The expression “lower alkyl” refers to alkyl groups composed of from 1 to 6 carbon atoms.

“Alkanyl” by itself or as part of another substituent refers to a saturated branched, straight-chain or cyclic alkyl derived by the removal of one hydrogen atom from a single carbon atom of a parent alkane. Alkanyl groups can include, but are not limited to, methanyl; ethanyl; propanyls such as propan-1-yl, propan-2-yl(isopropyl), cyclopropan-1-yl, etc.; butanyls such as butan-1-yl, butan-2-yl(sec-butyl), 2-methyl-propan-1-yl(isobutyl), 2-methyl-propan-2-yl(t-butyl), cyclobutan-1-yl, etc.; and the like.

“Alkenyl” by itself or as part of another substituent refers to an unsaturated branched, straight-chain or cyclic alkyl having at least one carbon-carbon double bond derived by the removal of one hydrogen atom from a single carbon atom of a parent alkene. The group may be in either the cis or trans conformation about the double bond(s). Alkenyl groups can include, but are not limited to, ethenyl; propenyls such as prop-1-en-1-yl, prop-1-en-2-yl, prop-2-en-1-yl, prop-2-en-2-yl, cycloprop-1-en-1-yl; cycloprop-2-en-1-yl; butenyls such as but-1-en-1-yl, but-1-en-2-yl, 2-methyl-prop-1-en-1-yl, but-2-en-1-yl, but-2-en-2-yl, buta-1,3-dien-1-yl, buta-1,3-dien-2-yl, cyclobut-1-en-1-yl, cyclobut-1-en-3-yl, cyclobuta-1,3-dien-1-yl, etc.; and the like.

“Alkynyl” by itself or as part of another substituent refers to an unsaturated branched, straight-chain or cyclic alkyl having at least one carbon-carbon triple bond derived by the removal of one hydrogen atom from a single carbon atom of a parent alkyne. Alkynyl groups can include, but are not limited to, ethynyl; propynyls such as prop-1-yn-1-yl, prop-2-yn-1-yl, etc.; butynyls such as but-1-yn-1-yl, but-1-yn-3-yl, but-3-yn-1-yl, etc.; and the like. In some embodiments, the alkynyl group is (C2-C6)alkynyl.

“Alkyleno” by itself or as part of another substituent refers to a straight-chain saturated or unsaturated alkyldiyl group having two terminal monovalent radical centers derived by the removal of one hydrogen atom from each of the two terminal carbon atoms of straight-chain parent alkane, alkene or alkyne. The locant of a double bond or triple bond, if present, in a particular alkyleno is indicated in square brackets. Typical alkyleno groups include, but are not limited to, methano; ethylenos such as ethano, etheno, ethyno; propylenos such as propano, prop[1]eno, propa[1,2]dieno, prop[1]yno, etc.; butylenos such as butano, but[1]eno, but[2]eno, buta[1,3]dieno, but[1]yno, but[2]yno, buta[1,3]diyno, etc.; and the like. Where specific levels of saturation are intended, the nomenclature alkano, alkeno and/or alkyno is used. For example, the alkyleno group can be (C1-C6) or (C1-C3)alkyleno. In some embodiments, the alkyleno group can be a straight-chain saturated alkano group, e.g., methano, ethano, propano, butano, and the like.

“Heteroalkyl,” Heteroalkanyl,” Heteroalkenyl,” Heteroalkynyl,” and “Heteroalkyleno” by themselves or as part of another substituent refer to alkyl, alkanyl, alkenyl, alkynyl, and alkyleno groups, respectively, in which one or more of the carbon atoms are each independently replaced with the same or different heteratoms or heteroatomic groups. Heteroatoms and/or heteroatomic groups which can replace the carbon atoms can include, but are not limited to, —O—, —S—, —S—O—, —NR′—, —PH—, —S(O)—, —S(O)₂—, —S(O)NR′—, —S(O)₂NR′—, and the like, including combinations thereof, where each R′ is independently hydrogen or (C1-C6)alkyl.

“Cycloalkyl” and “Heterocycloalkyl” by themselves or as part of another substituent refer to cyclic versions of “alkyl” and “heteroalkyl” groups, respectively. For heteroalkyl groups, a heteroatom can occupy the position that is attached to the remainder of the molecule. Cycloalkyl groups can include, but are not limited to, cyclopropyl; cyclobutyls such as cyclobutanyl and cyclobutenyl; cyclopentyls such as cyclopentanyl and cyclopentenyl; cyclohexyls such as cyclohexanyl and cyclohexenyl; and the like. Heterocycloalkyl groups can include, but are not limited to, tetrahydrofuranyl (e.g., tetrahydrofuran-2-yl, tetrahydrofuran-3-yl, etc.), piperidinyl (e.g., piperidin-1-yl, piperidin-2-yl, etc.), morpholinyl (e.g., morpholin-3-yl, morpholin-4-yl, etc.), piperazinyl (e.g., piperazin-1-yl, piperazin-2-yl, etc.), and the like.

“Parent Aromatic Ring System” refers to an unsaturated cyclic or polycyclic ring system having a conjugated it electron system. Specifically included within the definition of “parent aromatic ring system” are fused ring systems in which one or more of the rings are aromatic and one or more of the rings are saturated or unsaturated, such as, for example, fluorene, indane, indene, phenalene, tetrahydronaphthalene, etc. Parent aromatic ring systems can include, but are not limited to, aceanthrylene, acenaphthylene, acephenanthrylene, anthracene, azulene, benzene, chrysene, coronene, fluoranthene, fluorene, hexacene, hexaphene, hexylene, indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, tetrahydronaphthalene, triphenylene, trinaphthalene, and the like, as well as the various hydro isomers thereof.

“Aryl” by itself or as part of another substituent refers to a monovalent aromatic hydrocarbon group having the stated number of carbon atoms (i.e., C5-C15 means from 5 to 15 carbon atoms) derived by the removal of one hydrogen atom from a single carbon atom of a parent aromatic ring system. Aryl groups can include, but are not limited to, groups derived from aceanthrylene, acenaphthylene, acephenanthrylene, anthracene, azulene, benzene, chrysene, coronene, fluoranthene, fluorene, hexacene, hexaphene, hexylene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene, and the like, as well as the various hydro isomers thereof. In some embodiments, the aryl group is (C5-C15) aryl, or (C5-C10)aryl. Exemplary aryl groups are cyclopentadienyl, phenyl and naphthyl.

“Parent Heteroaromatic Ring System” refers to a parent aromatic ring system in which one or more carbon atoms are each independently replaced with the same or different heteroatoms or heteroatomic groups. Heteroatoms or heteroatomic groups to replace the carbon atoms can include, but are not limited to, N, NH, P, O, S, S(O), S(O)₂, Si, etc. Specifically included within the definition of “parent heteroaromatic ring systems” are fused ring systems in which one or more of the rings are aromatic and one or more of the rings are saturated or unsaturated, such as, for example, benzodioxan, benzofuran, chromane, chromene, indole, indoline, xanthene, etc. Also included in the definition of “parent heteroaromatic ring system” are those recognized rings that include substituents, such as benzopyrone. Parent heteroaromatic ring systems can include, but are not limited to, acridine, benzimidazole, benzisoxazole, benzodioxan, benzodioxole, benzofuran, benzopyrone, benzothiadiazole, benzothiazole, benzotriazole, benzoxaxine, benzoxazole, benzoxazoline, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like.

“Heteroaryl” by itself or as part of another substituent refers to a monovalent heteroaromatic group having the stated number of ring atoms (e.g., “5-14 membered” means from 5 to 14 ring atoms) derived by the removal of one hydrogen atom from a single atom of a parent heteroaromatic ring system. Heteroaryl groups can include, but are not limited to, groups derived from acridine, benzimidazole, benzisoxazole, benzodioxan, benzodiaxole, benzofuran, benzopyrone, benzothiadiazole, benzothiazole, benzotriazole, benzoxazine, benzoxazole, benzoxazoline, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like, as well as the various hydro isomers thereof. In some embodiments, the heteroaryl group is a 5-14 membered heteroaryl or 5-10 membered heteroaryl.

“Protecting Group” refers to a group of atoms that, when attached to a reactive functional group in a molecule, mask, reduce or prevent the reactivity of the functional group. A protecting group can be selectively removed as desired during the course of a synthesis. Examples of protecting groups can be found in Greene and Wuts, Protective Groups in Organic Chemistry, 3^{rd} Ed., 1999, John Wiley & Sons, NY and Harrison et al., Compendium of Synthetic Organic Methods, Vols. 1-8, 1971-1996, John Wiley & Sons, NY. Representative amino protecting groups include, but are not limited to, formyl, acetyl, trifluoroacetyl, benzyl, benzyloxycarbonyl (“CBZ”), tert-butoxycarbonyl (“Boc”), trimethylsilyl (“TMS”), 2-trimethylsilyl-ethanesulfonyl (“SES”), trityl and substituted trityl groups, allyloxycarbonyl, 9-fluorenylmethyloxycarbonyl (“FMOC”), nitro-veratryloxycarbonyl (“NVOC”) and the like. Representative hydroxyl protecting groups include, but are not limited to, those where the hydroxyl group is either acylated (e.g., methyl and ethyl esters, acetate or propionate groups or glycol esters) or alkylated such as benzyl and trityl ethers, as well as alkyl ethers, tetrahydropyranyl ethers, trialkylsilyl ethers (e.g., TMS or TIPPS groups) and allyl ethers.

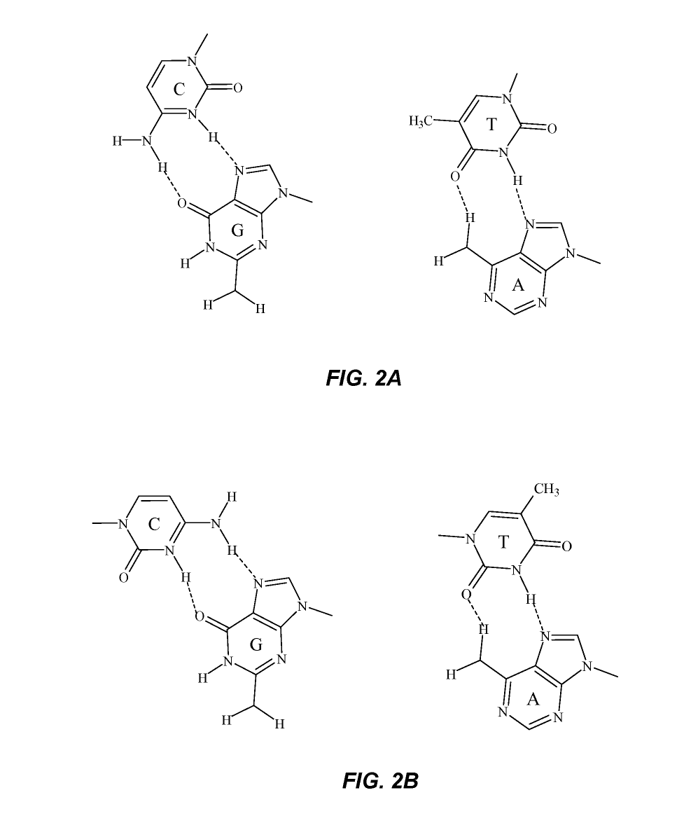

“Nucleobase” or “Base” refers to those naturally occurring and synthetic heterocyclic moieties commonly known to those who utilize nucleic acid or polynucleotide technology or utilize polyamide or peptide nucleic acid technology to thereby generate polymers that can hybridize to polynucleotides in a sequence-specific manner. Non-limiting examples of suitable nucleobases include: adenine, cytosine, guanine, thymine, uracil, 5-propynyl-uracil, 2-thio-5-propynyl-uracil, 5-methylcytosine, pseudoisocytosine, 2-thiouracil and 2-thiothymine, 2-aminopurine, N9-(2-amino-6-chloropurine), N9-(2,6-diaminopurine), hypoxanthine, isoguanine (iG), N9-(7-deaza-guanine), N9-(7-deaza-8-aza-guanine) and N8-(7-deaza-8-aza-adenine). Other non-limiting examples of suitable nucleobases include those nucleobases illustrated in FIGS. 2(A) and 2(B) of Buchardt et al. (WO 92/20702 or WO 92/20703).

“Nucleobase Polymer” or “Oligomer” refers to two or more nucleobases that are connected by linkages that permit the resultant nucleobase polymer or oligomer to hybridize to a polynucleotide having a complementary nucleobase sequence. Nucleobase polymers or oligomers include, but are not limited to, poly- and oligonucleotides (e.g., DNA and RNA polymers and oligomers), poly- and oligonucleotide analogs and poly- and oligonucleotide mimics, such as polyamide or peptide nucleic acids. Nucleobase polymers or oligomers can vary in size from a few nucleobases, from 2 to 40 nucleobases, 10 to 25 nucleobases, 12 to 30 nucleobases, or 12 to 20 nucleobases, to several hundred nucleobases, to several thousand nucleobases, or more.

“Polynucleotides” or “Oligonucleotides” refer to nucleobase polymers or oligomers in which the nucleobases are connected by sugar phosphate linkages (sugar-phosphate backbone). Exemplary poly- and oligonucleotides include polymers of 2′-deoxyribonucleotides (DNA) and polymers of ribonucleotides (RNA). A polynucleotide may be composed entirely of ribonucleotides, entirely of 2′-deoxyribonucleotides or combinations thereof.

“Polynucleotide Analog” or “Oligonucleotide Analog” refers to nucleobase polymers or oligomers in which the nucleobases are connected by a sugar phosphate backbone comprising one or more sugar phosphate analogs. Typical sugar phosphate analogs include, but are not limited to, sugar alkylphosphonates, sugar phosphoramidites, sugar alkyl- or substituted alkylphosphotriesters, sugar phosphorothioates, sugar phosphorodithioates, sugar phosphates and sugar phosphate analogs in which the sugar is other than 2′-deoxyribose or ribose, nucleobase polymers having positively charged sugar-guanidyl interlinkages such as those described in U.S. Pat. No. 6,013,785 and U.S. Pat. No. 5,696,253 (see also, Dagani 1995, Chem. Eng News 4-5:1153; Dempey et al., 1995, J Am Chem Soc 117:6140-6141). Such positively charged analogues in which the sugar is 2′-deoxyribose are referred to as “DNGs,” whereas those in which the sugar is ribose are referred to as “RNGs.” Specifically included within the definition of poly- and oligonucleotide analogs are locked nucleic acids (LNAs; see, e.g., Elayadi et al., 2002, Biochemistry 41:9973-9981; Koshkin et al., 1998, J Am Chem Soc 120:13252-3; Koshkin et al., 1998, Tetrahedron Letters 39:4381-4384; Jumar et al., 1998, Bioorg Med Chem Lett 8:2219-2222; Singh and Wengel, 1998, Chem Commun 12:1247-1248; WO 00/56746; WO 02/28875; and, WO 01/48190; all of which are incorporated herein by reference in their entireties).

“Polynucleotide Mimic” or “Oligonucleotide Mimic” refers to a nucleobase polymer or oligomer in which one or more of the backbone sugar-phosphate linkages is replaced with a sugar-phosphate analog. Such mimics are capable of hybridizing to complementary polynucleotides or oligonucleotides, or polynucleotide or oligonucleotide analogs or to other polynucleotide or oligonucleotide mimics, and may include backbones comprising one or more of the following linkages: positively charged polyamide backbone with alkylamine side chains as described in U.S. Pat. No. 5,786,461; U.S. Pat. No. 5,766,855; U.S. Pat. No. 5,719,262; U.S. Pat. No. 5,539,082 and WO 98/03542 (see also, Haaima et al., 1996, Angewandte Chemie Int'l Ed. in English 35:1939-1942; Lesnick et al., 1997, Nucleosid. Nucleotid. 16:1775-1779; D'Costa et al., 1999, Org. Lett. 1:1513-1516; and Nielsen, 1999, Curr Opin Biotechnol. 10:71-75); uncharged polyamide backbones as described in WO 92/20702 and U.S. Pat. No. 5,539,082; uncharged morpholino-phosphoramidate backbones as described in U.S. Pat. No. 5,698,685, U.S. Pat. No. 5,470,974, U.S. Pat. No. 5,378,841 and U.S. Pat. No. 5,185,144 (see also Wages et al., 1997, BioTechniques 23:1116-1121); peptide-based nucleic acid mimic backbones (see, e.g., U.S. Pat. No. 5,698,685); carbamate backbones (see, e.g., Stirchak and Summerton, 1987, J Org. Chem. 52:4202); amide backbones (see, e.g., Lebreton, 1994, Synlett. 1994:137); methylhydroxyl amine backbones (see, e.g., Vasseur et al., 1992, J Am Chem. Soc. 114:4006); 3′-thioformacetal backbones (see, e.g., Jones et al., 1993, J Org. Chem. 58:2983) and sulfamate backbones (see, e.g., U.S. Pat. No. 5,470,967). All of the preceding references are herein incorporated by reference for all purposes.

“Peptide Nucleic Acid” or “PNA” refers to poly- or oligonucleotide mimics in which the nucleobases are connected by amide linkages (i.e., polyamide backbone) such as described in any one or more of U.S. Pat. Nos. 5,539,082; 5,527,675; 5,623,049; 5,714,331; 5,718,262; 5,736,336; 5,773,571; 5,766,855; 5,786,461; 5,837,459; 5,891,625; 5,972,610; 5,986,053; 6,107,470; 6,451,968; 6,441,130; 6,414,112; and 6,403,763; all of which are incorporated herein by reference. The term “peptide nucleic acid” or “PNA” shall also apply to any oligomer or polymer comprising two or more subunits of those polynucleotide mimics described in the following publications: Lagriffoul et al., 1994, Bioorg Med Chem Lett 4: 1081-1082; Petersen et al., 1996, Bioorg Med Chem Lett 6: 793-796; Diderichsen et al., 1996, Tett Lett 37: 475-478; Fujii et al., 1997, Bioorg Med Chem Lett 7: 637-627; Jordan et al., 1997, Bioorg Med Chem Lett 7:687-690; Krotz et al., 1995, Tett Lett 36: 6941-6944; Lagriffoul et al, 1994, Bioorg Med Chem Lett 4:1081-1082; Diederichsen, U., 1997, Bioorg Med Chem Lett 7: 1743-1746; Lowe et al., 1997, J Chem Soc Perkin Trans 1:539-546; Lowe et al., 1997, J Chem Soc Perkin Trans 11:547-554; Lowe et al., 1997, J Chem Soc Perkin Trans 1:555-560; Howarth et al., 1997, J Org Chem 62:5441-5450; Altmann et al., 1997, Bioorg Med Chem Lett 7:1119-1122; Diederichsen, U., 1998, Bioorg Med Chem Lett 8:165-168; Diederichsen et al., 1998, Angew Chem Int Ed 37:302-305; Cantin et al., 1997, Tett Lett 38:4211-4214; Ciapetti et al., 1997, Tetrahedron 53:1167-1176; Lagriffoule et al., 1997, Chem Eur J 3:912-919; Kumar et al., 2001, Org Lett 3(9):1269-1272; and the Peptide-Based Nucleic Acid Mimics (PENAMs) disclosed in WO 96/04000. Some examples of PNAs are those in which the nucleobases are attached to an N-(2-aminoethyl)-glycine backbone, i.e., a peptide-like, amide-linked unit (see, e.g., U.S. Pat. No. 5,719,262; WO 92/20702; and Nielsen et al., 1991, Science 254:1497-1500). Other PNAs can have a cyclic backbone (see, e.g., U.S. Pat. No. 5,977,296; U.S. Pat. No. 6,716,961; WO 2004/0063906; and D'Costa et al., 1999, Org Lett 1(10):1513-6). All publications are incorporated herein by reference.

“Chimeric Nucleobase Polymer” or “Chimeric Oligo” refers to a nucleobase polymer or oligomer comprising a plurality of different polynucleotides, polynucleotide analogs and polynucleotide mimics. For example, a chimeric oligo may comprise a sequence of DNA linked to a sequence of RNA. Other examples of chimeric oligos include a sequence of DNA linked to a sequence of PNA, and/or a sequence of RNA linked to a sequence of PNA.

“Nucleoside” refers to a compound having a purine, deazapurine, or pyrimidine nucleoside base, e.g., adenine, guanine, cytosine, uracil, thymine, 7-deazaadenine, 7-deazaguanosine, that is linked to the anomeric carbon of a pentose sugar at the 1′ position, such as a ribose, 2′-deoxyribose, or a 2′,3′-di-deoxyribose. Unless otherwise stated, when the nucleoside base is purine or 7-deazapurine, the pentose is attached at the 9-position of the purine or deazapurine, and when the nucleoside base is pyrimidine, the pentose is attached at the 1-position of the pyrimidine (see, e.g., Kornberg and Baker, 1992, DNA Replication, 2nd Ed., Freeman). The term “nucleotide” as used herein refers to a phosphate ester of a nucleoside, e.g., a mono-, a di-, or a triphosphate ester, wherein the most common site of esterification is the hydroxyl group attached to the C-5 position of the pentose. “Nucleotide 5′-triphosphate” refers to a nucleotide with a triphosphate ester group at the 5′ position. The term “nucleoside/tide” as used herein refers to a set of compounds including both nucleosides and/or nucleotides.

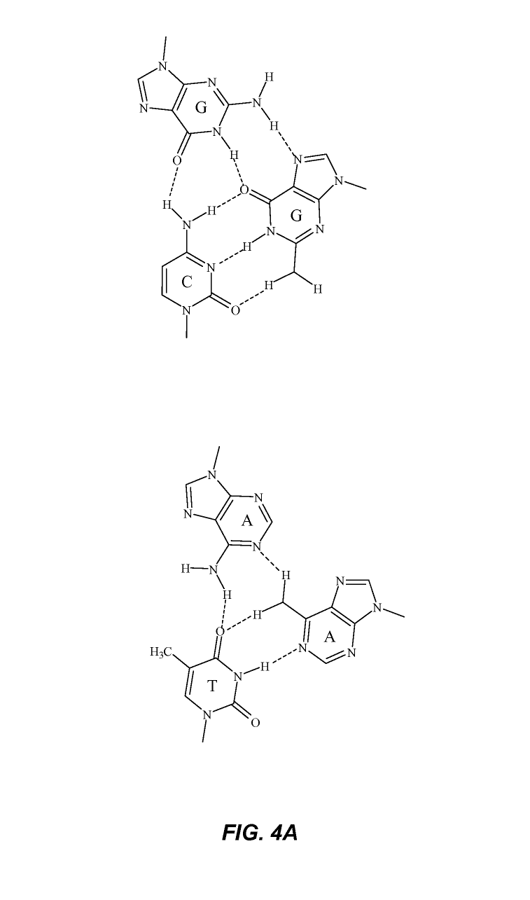

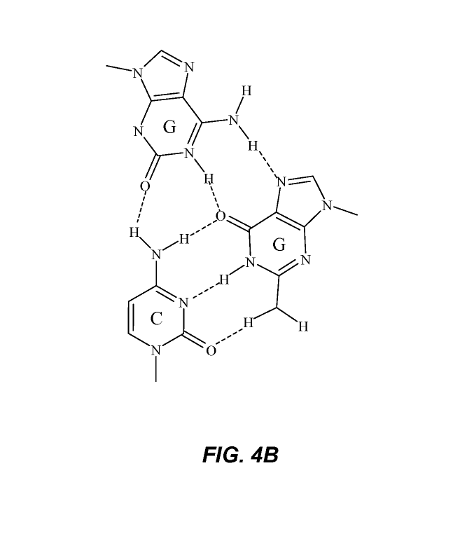

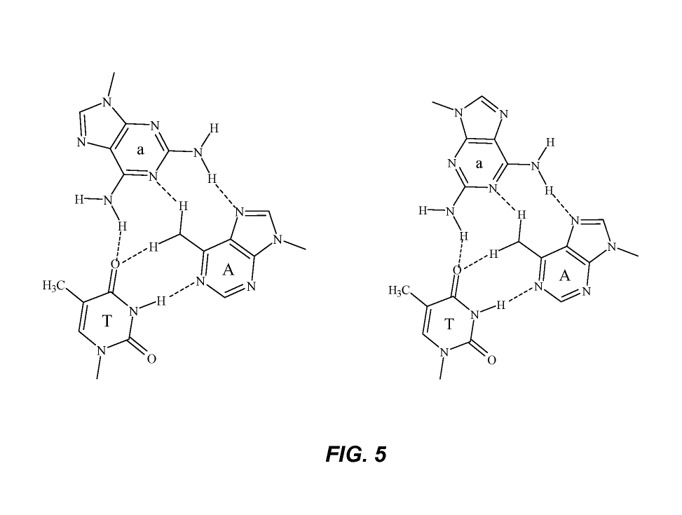

Annealing” or “Hybridization” refers to the base-pairing interactions of one nucleobase polymer with another that results in the formation of a double-stranded structure, a triplex structure, or other multi-stranded secondary structures, including hairpin type structures formed by a self complementary nucleobase polymer. Annealing or hybridization can occur via Watson-Crick base-pairing interactions, but may be mediated, in part, by other hydrogen-bonding interactions, such as Hoogsteen and Reverse Hoogsteen hydrogen bonding, and Straus-Matysiak and Reverse Straus-Matysiak hydrogen bonding (see FIGS. 4 and 5).

“Hydrogen Bond Donor” refers to an electronegative atom or group of atoms with a covalently linked hydrogen atom.

“Hydrogen Bond Acceptor” refers to an atom or group of atoms that is attracted to a hydrogen atom of a hydrogen bond donor.

“Hydrogen Bond” refers to a bond formed between a hydrogen bond acceptor and the hydrogen atom of a hydrogen bond donor.

“Detectable Label” refers to a moiety that can be detected using known detection methods, e.g., spectroscopic, photochemical, electrochemiluminescent, and enzymatic, when the label is attached to a compound or composition to be detected. Exemplary labels include, but are not limited to, fluorophores, lumiphores, and radioisotopes.

“Substrate,” “Support,” “Solid Support,” “Solid Carrier,” or “Resin” are interchangeable terms and refer to any solid phase material. Substrate also encompasses terms such as “solid phase,” “surface,” and/or “membrane.” A solid support can be composed of organic polymers such as polystyrene, polyethylene, polypropylene, polyfluoroethylene, polyethyleneoxy, and polyacrylamide, as well as co-polymers and grafts thereof. A solid support can also be inorganic, such as glass, silica, controlled pore glass (CPG), reverse phase silica or metal, such as gold or platinum. The configuration of a substrate can be in the form of beads, spheres, particles, granules, a gel, a membrane or a surface. Surfaces can be planar, substantially planar, or non-planar. Solid supports can be porous or non-porous, and can have swelling or non-swelling characteristics. A solid support can be configured in the form of a well, depression, or other container, vessel, feature, or location. A plurality of supports can be configured on an array at various locations, addressable for robotic delivery of reagents, or by detection methods and/or instruments.

4.2 Compositions of Triplexes

Triple-stranded or triplex forms of polynucleotides have been described for those formed between a poly(purine:pyrimidine) tract in a Watson-Crick base-paired duplex and a third nucleobase polymer strand having a tract of polypyrimidines or polypurines. As described above, in one type of triplex structure, a polypyrimidine binds parallel to the purine strand of a Watson Crick base-paired polypyrimidine:polypurine duplex to form C:G*C⁺and T:A*T triplexes (C⁺=protonated cytosine) via Hoogsteen hydrogen bonding. In the second type of triplex structure, a G-rich polypurine binds antiparallel to the purine strand of a Watson-Crick base-paired polypyrimidine:polypurine duplex to form C:G*G and T:A*T or C:G*G and T:A*A triplexes via reverse Hoogsteen hydrogen bonding.

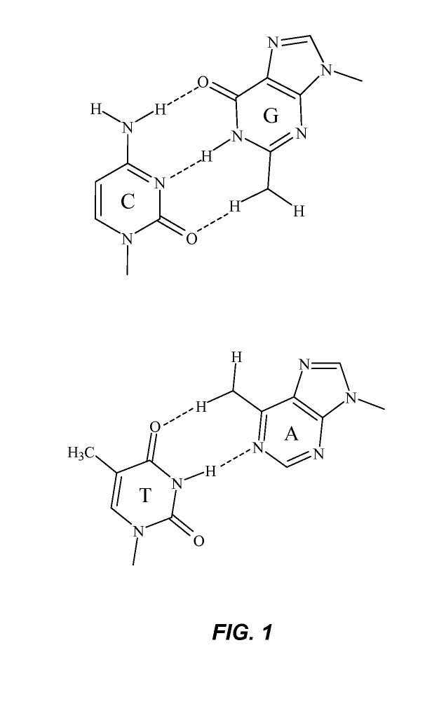

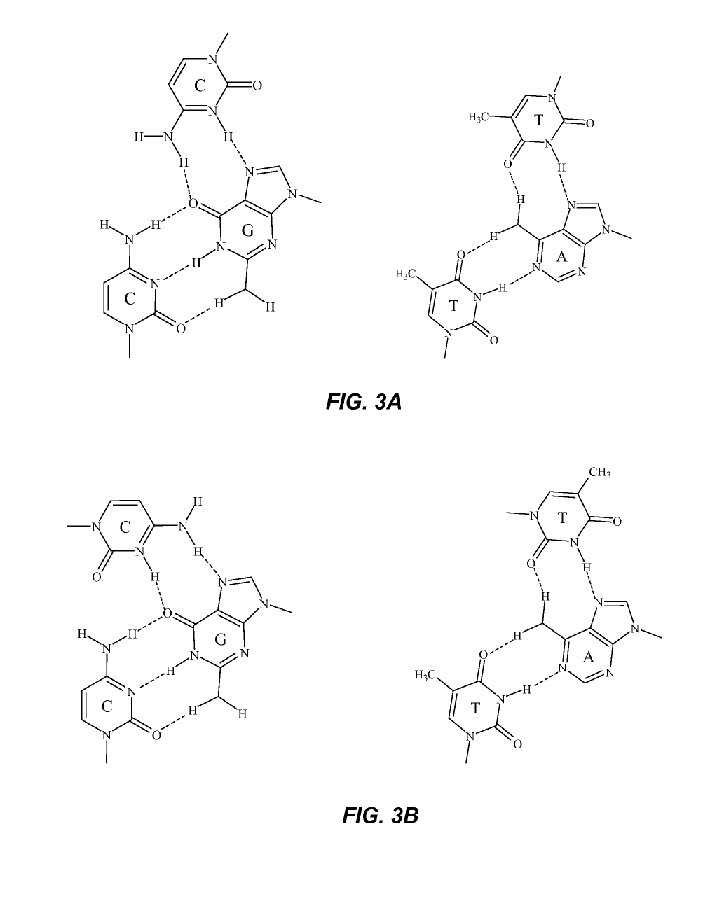

The Watson-Crick hydrogen bonding and the Hoogsteen/Reverse Hoogsteen hydrogen bonding used in the two types of triplex structures are illustrated in FIG. 1 and FIG. 2, respectively. The hydrogen bonding schemes in the triplex structures of the pyrimidine motif are shown in FIG. 3A and FIG. 3B. In either of the known triplex structures, the polypyrimidine or purine base of the triplex forming oligonucleotide is hydrogen bonded with only one strand of the duplex, e.g., the strand with the tract of polypurines.

In the present disclosure, a hydrogen bonding scheme has been identified from modeling studies, different from the typical Hoogsteen/Reverse Hoogsteen hydrogen bonding used in known triplex structures, that can be incorporated to form triplex structures. Double-stranded DNA in the B-form is a helical structure having a major groove and a minor groove formed between the backbones of the polynucleotides. The larger major groove is wide and deep while the minor groove is narrow and shallow. Although both the major groove and the minor groove have potential hydrogen bond acceptors and donors, the pattern of possible hydrogen bonds is more specific and discriminatory in the major groove than the minor groove. Moreover, the major groove is wider, thus making the hydrogen bonding more accessible for interaction with other molecules, such as DNA binding proteins. The informational content of the major groove makes the major groove generally the site of direct information readout. For a GC pair, the potential hydrogen bond formation is an acceptor, an acceptor, and a donor from the G to C direction in the groove. The arrangement of a CG pair is a donor, acceptor, and acceptor. For the AT pair, the potential hydrogen bond formation is an acceptor, donor, acceptor from A to T direction in the groove. The TA pair then has the order acceptor, donor, and acceptor. The positioning of the potential hydrogen bonds in the major groove also differs with the specific base-pairs: GC, CG, AT, and TA. The different ring structures of purines and pyrimidines result in distinctly different positioning of potential hydrogen bond formers in the major groove. In GC and AT base-pairs, the first potential hydrogen bond on the purine side of the major groove is a nitrogen in the small ring of these purines; this nitrogen is only one carbon away from the nitrogen that attaches the backbone to the base. In the CG and TA base-pairs, the first potential hydrogen bond former on the left side or the pyrimidine side of the major groove is three carbon atoms away from the nitrogen that attaches the base to the backbone. This means that the cluster of potential hydrogen bond formers is either on one side or the other of the major groove. It is this combination of different potential hydrogen bond formation and position of these hydrogen bonds that give specific base-pairs their own unique informational content to the major groove. This provides the basis for the specific binding between protein repressors and the genes they repress. It also is the basis for the recognition between restriction enzymes and the specific sequences they cleave.

As disclosed herein, the inventors have used the informational content of hydrogen bond formers of the major groove and identified specific hydrogen bonding patterns useful for generating triplex structures that do not rely on typical Hoogsteen/Reverse Hoogsteen hydrogen bonding. This hydrogen bonding scheme disclosed herein is referred to as the “Straus-Matysiak” hydrogen bonding in which a purine base of a third strand forms hydrogen bonds with both purine and pyrimidine bases of a purine:pyrimidine base-pair in a duplex formed between two nucleobase polymers. The specifics of Straus-Matysiak hydrogen bonding scheme is illustrated in FIGS. 4A-4C for purine base on a TFNP and a purine:pyrimidine base-pair of a Watson-Crick base-paired duplex. Anti-conformations are possible for some types of purine bases, such as isoguanine and 2,6-diaminopurines (see FIG. 4B and FIG. 5, respectively). Because the Straus-Matysiak hydrogen bonding occurs through the major groove of a duplex, a nucleobase polymer with bases hydrogen bonding through the Straus-Matysiak pattern can be accommodated in the major groove as a third strand of a triplex. As will be apparent to the skilled artisan, the triplex nucleobase polymers based on the Straus-Matysiak hydrogen bonding differ from triplex nucleobase polymers based on Hoogsteen hydrogen bonding in at least two respects: (1) the triplex nucleobase polymer has a polypurine tract with purines bases complementary to the pyrimidine bases of the pyrimidine:purine tract of the duplex strands; and (2) the purines of the third strand hydrogen bond to both strands of the duplex to form the triplex structure.

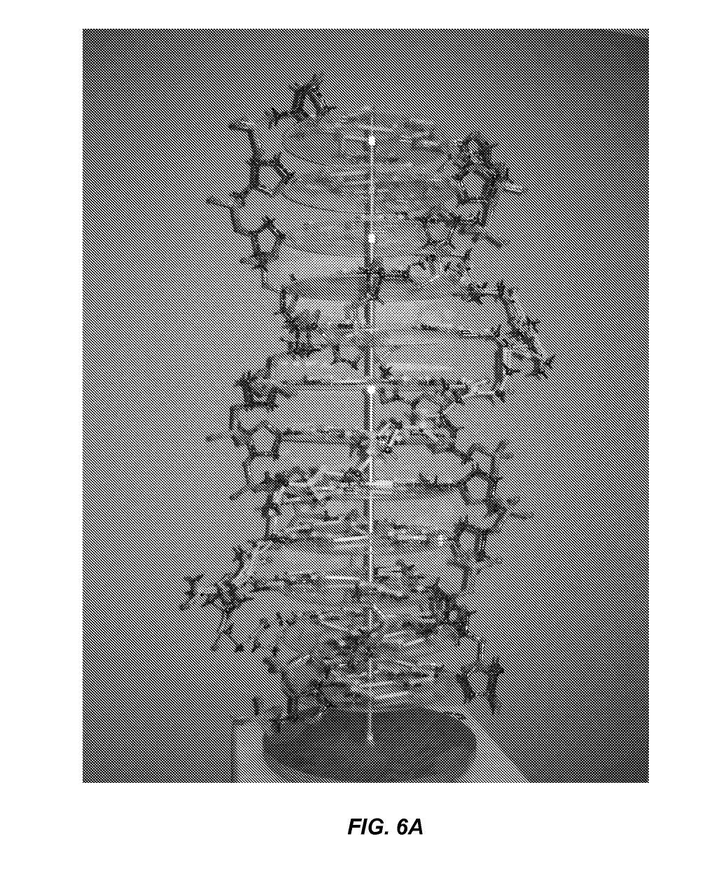

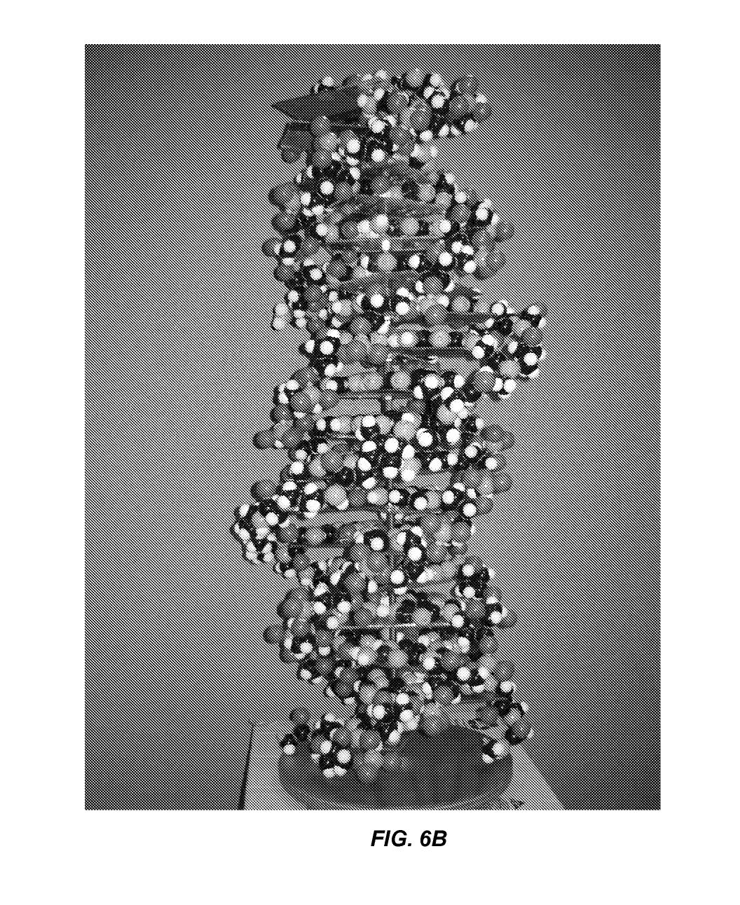

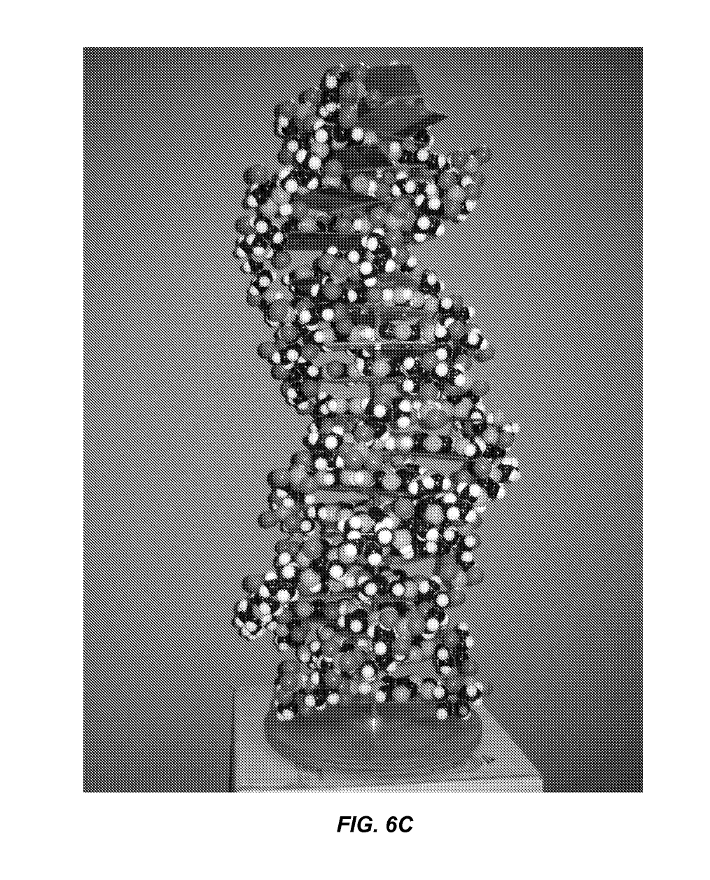

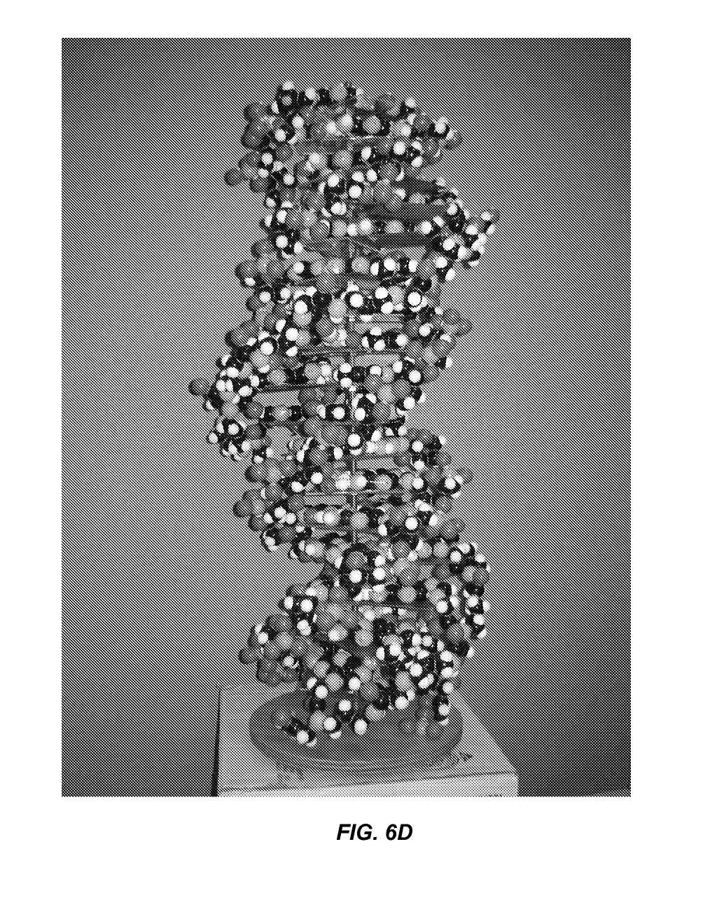

Model building studies based on the Straus-Matysiak hydrogen bonding shows that the two-ringed structures of purines place the nucleobase polymer backbone at a more distant position from the axis of the duplex as compared to the backbones of a duplex formed by typical sugar-phosphate backbones in B-form DNA (FIG. 6). To accommodate this additional base-to-base distance imposed by this hydrogen bonding scheme of the purine base, the backbone can be suitably extended by insertion of additional atoms into the backbone of a subunit of the nucleobase polymer. An added advantage is that the constraints imposed by the extended backbone is likely to limit the annealing of the TFNP with the strands forming the Watson-Crick base-paired duplex. This would attenuate the formation of the strand invasion complexes typical of triplex structures, such as the P-looped structures generated by a polypyrimidine PNAs with N-(2-aminoethyl)glycine backbones (i.e., PNA:DNA*PNA+DNA). Use of an uncharged backbone or a backbone with one or more positive charges can reduce any charge-charge repulsion present when the backbone of the duplex strands are composed of negatively charged sugar-phosphate groups.

In accordance with the above, the present disclosure provides compositions of triplex structures formed using Straus-Matysiak hydrogen bonding patterns. Generally, the composition of triplex comprises a polypurine tract and a polypyrimidine tract complementary to, and annealed to, each other, thereby forming a duplex segment of poly(purine:pyrimidine) base-pairs. A second polypurine tract complementary to the polypyrimidine tract is hydrogen bonded to both the polypurine and polypyrimidine tracts of the duplex segment. Thus, each purine base of the second polypurine tract can be hydrogen bonded to both purine and pyrimidine bases of a poly(purine:pyrimidine) base-pair of the duplex segment. In various embodiments, the duplex segment of the polypurine and polypyrimidine is hydrogen bonded by Watson-Crick hydrogen bonding while the second polypurine tract is hydrogen bonded to the polypurine and polypyrimidine tracts through the Straus-Matysiak hydrogen bonding.

A “polypurine tract” refers to a contiguous segment of the nucleobase polymer in which the bases can be all or substantially all purines. Similarly, a “polypyrimidine tract” refers to a contiguous segment of the nucleobase polymer in which the bases can be all or substantially all polypyrimidines. In some embodiments, the polypurine tract comprises all purines while the polypyrimidine tract comprise all pyrimidines. The polypurine tracts and its complementary polypyrimidine tracts on the duplex segment can be sufficiently long to allow formation of a triplex structure with the second polypurine tract.

In various embodiments, the polypurine tract, the polypyrimidine tract, and the second polypurine tract forming the triplex composition can be all on a single strand, or on different strands of nucleobase polymers. In an exemplary embodiment, the polypurine tract, the polypyrimidine tract, and the second polypurine tract are all on a single strand of a polymer. Presence of intervening sequences or linkers between the polypurine and polypyrimidine tracts can permit folding of the single-stranded nucleobase polymer to accommodate specific hydrogen bonding interactions that lead to formation of a triplex structure. In other embodiments, the polypurine tract and the polypyrimidine tract can be on a single strand of a nucleobase polymer while the second polypurine tract can be on a separate nucleobase polymer strand. Folding of the nucleobase polymer with the polypurine and polypyrimidine tract forms the duplex segment of poly(purine:pyrimidine) while the nucleobase polymer strand with the second polypurine tract hydrogen bonds to the duplex segment of poly(purine:pyrimidine) to generate the triplex. In some embodiments, the triplex structure can be formed from separate nucleobase polymer strands, for example, a first separate strand comprising the polypurine tract, a second separate strand comprising the polypyrimidine tract, and a third separate strand comprising the second polypurine tract.

Thus, in some embodiments, the present disclosure provides compositions comprising a first nucleobase polymer comprising a polypurine tract and a second nucleobase polymer comprising a polypyrimidine tract that is complementary to, and annealed to, the polypurine tract of the first nucleobase polymer, thereby forming a duplex comprising a duplex segment of poly(purine:pyrimidine) base-pairs. A third nucleobase polymer, also referred to herein as the “TFNP,” with a second polypurine tract that is complementary to the polypyrimidine tract of the duplex segment, comprises a backbone of sufficient length such that the second polypurine tract is hydrogen bonded to both polypurine and polypyrimidine tracts of the duplex segment. In these embodiments, each purine base of the second polypurine tract is hydrogen bonded to both purine and pyrimidine bases of a purine:pyrimidine base-pair of the duplex segment.

The length of the polypurine tract and corresponding polypyrimidine tract on the first nucleobase and second nucleobase polymer, respectively, can be of any length sufficient to allow formation of a stable triplex with the polypurine tract of the third nucleobase polymer. In some embodiments, the polypurine tract and corresponding polypyrimidine tract can be about 5, 10, 15, 20, 30, 40, 50, 75, 100, 150 or 200 nucleobases or longer. As such, the corresponding polypurine tract of the third nucleobase polymer can be about 5, 10, 15, 20, 30, 40, 50, 75, 100, or 200 nucleobases or longer. For example, the polypurine tract of the third nucleobase polymer can comprise from about 5 to about 30 nucleobases, from about 7 to about 25 nucleobases, from about 10 to about 18 nucleobases or from about 12 to about 16 nucleobases.

In some embodiments, the triplex compositions are of the structural formula (I):

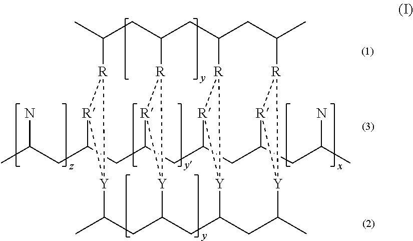

wherein:
(1) is a first nucleobase polymer;
(2) is a second nucleobase polymer; and
(3) is a third nucleobase polymer;

wherein:
each dashed line represents one or more hydrogen bonds between the nucleobases of the first, second, and third nucleobase polymers;
each

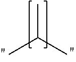

represents a backbone moiety of a subunit of each nucleobase polymer;
each N is, independently of the others, a nucleobase;
each R is, independently of the others, a purine nucleobase;
each Y is a pyrimidine nucleobase that is complementary to the R purine nucleobase to which it is hydrogen bonded;
each R′ is a purine nucleobase that is complementary to the Y pyrimidine nucleobase to which it is bonded;
x is an integer ranging from 0 to 50;
y is an integer ranging from 2 to 30;
y′ is an integer ranging from 2 to 30; and
z is an integer ranging from 0 to 50.

In some embodiments, y=y′ such that the length in bases of the duplex poly(purine:pyrimidine) segment can be the same for the length in bases of the polypurine tract of the third nucleobase polymer.

In the triplex of structural formula (1), the first and second nucleobase polymers are not limited with respect to the number of nucleobase polymer subunits that can be present at the ends of the polymers. In some embodiments, the first and/or second nucleobase polymers can have at one or both ends:

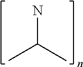

where each “n”, independently of the others, can be 1 or greater, and can be up to 5, 10, 20, 50, 100, 200, 500, or 1000 or more. Thus, in some embodiments, the first and/or second nucleobase polymers can be part of longer nucleobase polymers, as further described below.

In some embodiments, the purine base of the second polypurine tract of the third nucleobase polymer can be selected from adenine, guanine, 2,6-diaminopurine, and isoguanine. In some embodiments, for the triplex structure of structural formula (I):

each N can be, independently of the others, selected from adenine, cytosine, guanine, thymine, and uracil;

each R can be, independently of the others, selected from adenine, guanine, and 2,6-diaminopurine;

each Y can be, independently of the others, selected from cytosine, thymine, uracil, 2-thiouracil, 2-thiothymine, and pseudoisocystine; and

each R′ can be, independently of the others, selected from adenine, guanine, 2,6-diaminopurine, and isoguanine.

It is to be understood, however, that other purines and pyrimidines with the equivalent hydrogen bonding donor and acceptor patterns and geometries can be used. In addition, because of the specific hydrogen bonding patterns, the sequence of the duplex segment can determine the selection of R′ and vice versa.

As noted above, in various embodiments, the backbone of the second polypurine tract on the TFNP is of sufficient length such that each purine base of the second polypurine tract forms hydrogen bonds with both purine and pyrimidine bases of a purine:pyrimidine base-pair of the duplex segment. It is shown from modeling studies that extending the backbone of each subunit of the second polypurine tract on the TFNP by the inclusion of a carbon atom can accommodate the increased base-to-base distance imposed by the Straus-Matysiak hydrogen bonding disclosed herein. For a PNA, the carbon atom can be inserted into the N-(2-aminoethyl)glycine backbone typically used in classical PNA polymers. Exemplary embodiments of PNA backbones with extended backbones include, among others, a PNA with an N-(2-aminoethyl)-β-alanine backbone and a PNA with an N-(3-aminopropyl)glycine backbone (See structures 3 and 2, respectively of Table 1 of Nielsen et al., 1997, “Peptide nucleic acid (PNA). A DNA mimic with a pseudopeptide backbone,” Chemical Society Reviews pp. 73-78). In various embodiments, any nucleobase polymer comprising a backbone of appropriate length in which the backbone is uncharged or comprises one or more positive charges can be used to form the triplex structures. For example, the nucleobase polymer can be a polynucleotide analog or a polynucleotide mimic. Without limitations by theory, the use of neutral or positively charged backbones can minimize charge-charge repulsion that can be present if negatively charged backbones form the duplex segment to which the TFNP hydrogen bonds.

In accordance with the foregoing, in some embodiments, the triplex structure of formula (I) can comprise a third nucleobase polymer in which each subunit

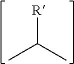

of the third nucleobase polymer represents a group according to structural formula (II):

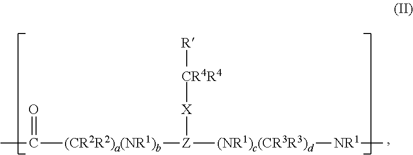

wherein:
each R¹ is independently H or lower alkyl;
each R² is independently H, lower alkyl, or alkylamine;
each R³ is independently H or lower alkyl;
each R⁴ is independently H or lower alkyl;
a is 1, 2 or 3;
b is 0 or 1;
c is 0 or 1;
d is 1, 2, or 3;
Z is —CR¹— or N, wherein R¹ is defined as above;
X is —CR⁵R⁵—, —C(O)—, —C(S), or —NR′—, wherein R¹ is defined as above, and each R⁵ is independently H or lower alkyl;
a+b+c+d=4; or

optionally wherein:
b and c is 0, a is 1, d is 3, and
(i) R² and R⁴ together with Z and X;
(ii) R³ and R⁴ together with Z and X;
(iii) R² and R³ together with Z;
(iv) R² with Z; or
(v) R³ with Z
is a five or six membered cycloalkyl or heterocycloalkyl ring.

In some embodiments, the PNA of structural formula (II) can be a nucleobase polymer in which Z is N and X is —C(O)—.

In some embodiments, the PNA of structural formula (II) can be a nucleobase polymer in which Z is N, each R⁴ is H, and X is —CR⁵R⁵—, wherein each R⁵ is H.

In some embodiments, the PNA structure of structural formula (II) can be a nucleobase polymer in which “a” is 1 and “d” is 3.

In some embodiments, the PNA structure of structural formula (II) can be a nucleobase polymer in which “a” is 2 and “d” is 2.

It is to be understood that other combinations of “a”, “b”, “c” and “d” can be used to form the extended backbone sufficient to permit hydrogen bonding patterns of the third strand as described herein.

In some embodiments, the PNA of structural formula (II) can comprise a nucleobase polymer in which each

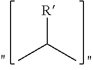

of the third nucleobase polymer represents a subunit according to structural formula (III):

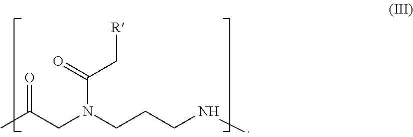

In some embodiments, the PNA of structural formula (II) can comprise a nucleobase

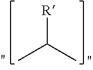

polymer in which each in the third nucleobase polymer represents a subunit according to structural formula (IV):

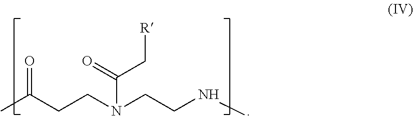

In some embodiments, the PNA of structural formula (II) can comprise a nucleobase polymer in which each

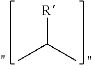

in the third nucleobase polymer represents a subunit according to structural formula (V):

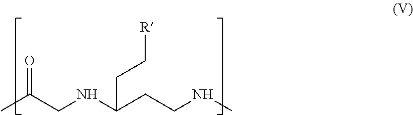

In some embodiments, the PNA of structural formula (II) can comprise a nucleobase polymer in which each

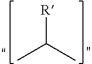

in the third nucleobase polymer represents a subunit having a cyclic backbone. In some embodiments, the subunit comprising the cyclic backbone can be selected from:

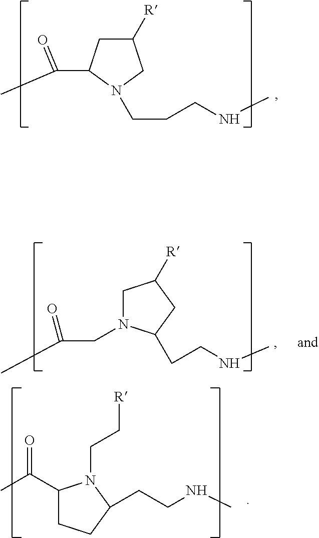

In various embodiments, the duplex segment of the triplex can comprise Watson-Crick hydrogen bonded nucleobase polymers. In some embodiments, the first and second nucleobase polymers can comprise sugar-phosphate backbones, or backbones that are analogs or mimics of sugar phosphate backbones. In some embodiments, one or both of the nucleobase polymers of the duplex can have a backbone of a 2′-deoxyribophosphate. In some embodiments, one or both of the nucleobase polymers of the duplex can have a backbone of a 2′-ribophosphate.

As will be apparent to the skilled artisan, various combinations of nucleobase polymers having deoxyribophosphate and/or ribophosphate backbones can be used to form the duplex. Exemplary embodiments include, among others, a first and second nucleobase polymer of 2′-deoxyribophosphates, and first nucleobase polymer of deoxyribophosphate and a second nucleobase polymer of ribophosphate, and a first nucleobase polymer of ribophosphate and a second nucleobase polymer of deoxyribophosphate.

In some embodiments where the first and/or second nucleobase polymer comprise a sugar-phosphate backbone, the attachment of the nucleobase to the sugar moiety of the sugar-phosphate backbone of each strand, can be independently of the other, in the α or β conformation. In some embodiments, both polynucleotides forming the duplex can be either in the α or β conformation to generate duplexes having anti-parallel strands. In some embodiments, one polynucleotide polymer can be in the α conformation and the other polynucleotide in the β conformation to form duplexes in the parallel conformation.

As will be apparent to the skilled artisan, the third nucleobase polymer can have different orientations with respect to the strands of the duplex, such as, for example, relative to the type of backbones on one or both strands of the duplex. In embodiments where the third nucleobase polymer is a PNA, the TFNP can be in two different orientations in the triplex with respect to a polypurine tract in the duplex segment for those embodiments where the polypurine tract has a sugar-phosphate backbone. In some embodiments, the TFNP can have its amino terminus oriented towards the 5-prime terminus of the polypurine tract. In the present disclosure, this orientation is referred to as the “parallel” orientation. In some embodiments, the third strand can have its carboxy terminus oriented towards the 3-prime terminus of the polypurine tract. In the present disclosure, this orientation is referred to as the “anti-parallel” orientation. As will be apparent to the skilled artisan, other types of TFNPs with different backbones can have analogous orientations with respect to the strand with the polypurine tract in the Watson-Crick base-paired duplex.

It is to be understood that the triplexes of the present disclosure are intended to include those in which the third nucleobase polymer is annealed to more than one duplex segment. In these embodiments, two or more of the complementary polypyrimidine tracts can be on the same strand or on different strands of the duplex. In some embodiments, the polypurine tracts of the TFNP can be connected to each other through a linker of sufficient length to permit hydrogen bonding of the TFNP to the duplex segments.

Accordingly, in some embodiments, the disclosure provides a triplex comprising: a first nucleobase polymer comprising a plurality of polypurine tracts; a second nucleobase polymer comprising a plurality of polypyrimidine tracts complementary to, and annealed to, the first plurality of polypurine tracts, thereby forming a duplex comprising a plurality of duplex segments of poly(purine:polypyrimidine) base-pairs; and a third nucleobase polymer comprising a second plurality of polypurine tracts complementary to the plurality of polypyrimidine tracts of the duplex segments. As described herein, the third nucleobase polymer comprises a backbone of sufficient length such that each purine base of the plurality of polypurine tracts on the third nucleobase polymer is hydrogen bonded to both purine and pyrimidine bases of a purine:pyrimidine base-pair of the plurality of duplex segments. In some embodiments, the polypurine tracts of the third nucleobase polymer can be connected to each other through a segment of a nucleobase polymer. In some embodiments, the polypurine tracts of the third nucleobase polymer can be connected to each other by a linker of sufficient length such that each purine base of the TNFP can be hydrogen bonded to both purine and pyrimidine bases of a purine:pyrimidine base-pair in the plurality of duplex segments.

Thus, in some embodiments, the triplex can comprise: a first nucleobase polymer comprising a first two polypurine tracts; a second nucleobase polymer comprising two polypyrimidine tracts complementary to, and annealed to, the first two polypurine tracts, thereby forming a duplex of a first and second duplex segments of poly(purine:polypyrimidine) base-pairs; and a third nucleobase polymer comprising a second two polypurine tracts complementary to the two polypyrimidine tracts of the first and second duplex segments. In these embodiments, the third nucleobase polymer can comprise a backbone of sufficient length and a linker connecting the second two polypurine tracts to each other such that each purine base of the second two polypurine tracts can be hydrogen bonded to both purine and pyrimidine bases of a purine:pyrimidine base-pair of the first or second duplex segments.

In some embodiments, where complementary polypyrimidine tracts are on different strands of the duplex, the triplex can comprise: a first nucleobase polymer comprising a first plurality of polypurine tract and polypyrimidine tract; a second nucleobase polymer comprising a second plurality of polypyrimidine tract and polypurine tract complementary to, and annealed to, the first plurality of polypurine tract and polypyrimidine tract of the first nucleobase polymer, thereby forming a duplex comprising a plurality of duplex segments of poly(purine:pyrimidine) base-pairs and poly(pyrimidine:purine) base-pairs; and a third nucleobase polymer comprising a plurality of polypurine tracts complementary to the polypyrimidine tracts of the plurality of duplex segments. In these embodiments, the third nucleobase polymer comprises a backbone of sufficient length such that each purine base of the plurality of polypurine tracts of the third nucleobase polymer can be hydrogen bonded to both purine and pyrimidine bases of a purine:pyrimidine base-pair or a pyrimidine:purine base-pair in the plurality of duplex segments. As above, in some embodiments, the polypurine tracts of the third nucleobase polymer can be connected to each other through a segment of a nucleobase polymer. In some embodiments, the polypurine tracts of the third nucleobase polymer can be connected to each other by a linker of sufficient length such that each purine base of the TFNP can be hydrogen bonded to both purine and pyrimidine bases of a purine:pyrimidine base-pair or a pyrimidine:purine base-pair in the plurality of duplex segments.

Thus, in some embodiments, a triplex can comprise a first nucleobase polymer comprising a first polypurine tract and first polypyrimidine tract; a second nucleobase polymer comprising a second polypyrimidine tract and second polypurine tract complementary to, and annealed to, the first polypurine tract and first polypyrimidine tract of the first nucleobase polymer, thereby forming a duplex comprising a first duplex segment of poly(purine:pyrimidine) base-pairs and a second duplex segment of poly(pyrimidine:purine) base-pairs; and a third nucleobase polymer comprising two polypurine tracts complementary to the first and second polypyrimidine tracts of the first and second duplex segments. In these embodiments, the third nucleobase polymer can comprise a backbone of sufficient length and a linker connecting the two polypurine tracts to each other such that each purine base of the two polypurine tracts can be hydrogen bonded to the purine and pyrimidine bases of a purine:pyrimidine base-pair of the first duplex segment or a pyrimidine:purine base-pair of the second duplex segment.

Linkers connecting the polypurine tracts can be any linker that allows formation of the hydrogen bonds with the duplex segments to form the triplex (see, e.g., WO 96/02558). As used herein, a “linker” refers to a chemical moiety comprising a covalent bond or a chain of atoms that connects two molecules. In the present disclosure, molecules include nucleobase polymers such as those containing the polypurine tracts. The linker can be selected to have specified properties. For example, the linker can be hydrophobic in character, hydrophilic in character, long or short, rigid, semi-rigid or flexible, depending upon the particular application. The linker can be optionally substituted with one or more substituents or one or more linking groups for the attachment of additional substituents, which may be the same or different, thereby providing a “polyvalent” linking moiety capable of conjugating or linking additional molecules or substances. In certain embodiments, however, linker does not comprise such additional substituents or linking groups.

A wide variety of linkers comprised of stable bonds are known in the art, and include by way of example and not limitation, alkyldiyls, substituted alkyldiyls, alkylenos (e.g., alkanos), substituted alkylenos, heteroalkyldiyls, substituted heteroalkyldiyls, heteroalkylenos, substituted heteroalkylenos, acyclic heteroatomic bridges, aryldiyls, substituted aryldiyls, arylaryldiyls, substituted arylaryldiyls, arylalkyldiyls, substituted arylalkyldiyls, heteroaryldiyls, substituted heteroaryldiyls, heteroaryl-heteroaryl diyls, substituted heteroaryl-heteroaryl diyls, heteroarylalkyldiyls, substituted heteroarylalkyldiyls, heteroaryl-heteroalkyldiyls, substituted heteroaryl-heteroalkyldiyls, and the like. Thus, a linker can include single, double, triple or aromatic carbon-carbon bonds, nitrogen-nitrogen bonds, carbon-nitrogen bonds, carbon-oxygen bonds, carbon-sulfur bonds, oxygen-oxygen bonds, and combinations of such bonds, and may therefore include functionalities such as carbonyls, ethers, thioethers, carboxamides, sulfonamides, ureas, urethanes, hydrazines, etc. In some embodiments, the linker can have from 1-20 non-hydrogen atoms selected from the group consisting of C, N, O, and S and can be composed of any combination of ether, thioether, amine, ester, carboxamide, sulfonamides, hydrazide, aromatic and/or heteroaromatic groups.

Choosing a linker having properties suitable for a particular application is within the capabilities of those having skill in the art. For example, where a rigid linker is desired, the linker can comprise a rigid polypeptide such as polyproline, a rigid polyunsaturated alkyldiyl or an aryldiyl, biaryldiyl, arylarydiyl, arylalkyldiyl, heteroaryldiyl, biheteroaryldiyl, heteroarylalkyldiyl, heteroaryl-heteroaryldiyl, etc. Hydrophilic linkers may comprise, for example, polyalcohols or polyethers such as polyalkyleneglycols (e.g., 8-amino-3,6-dioxaoctanoic acid and linkers based upon the PNA backbone, for example, as described in Gildea et al., 1998, Tett Lett 39:7255-7258; and U.S. Pat. Nos. 6,326,479 and 6,770,442). Hydrophobic linkers may comprise, for example, alkyldiyls or aryldiyls. In some embodiments, where a flexible linker is desired, the linker can comprise a flexible polypeptide such as polyglycine or a flexible saturated alkanyldiyl or heteroalkanyldiyl. Exemplary flexible linkers can be based on polyethylene glycol, ethylene glycol-phosphates, 3-hydroxypropane-1-phosphates (see, e.g., Pils et al., 2000, Nucleic Acids Res 28(9):1859-1863; Nulf et al., 2002, Nucleic Acids Res 30(13):2782-2789); polyamides, including D, L, and DL amino acids of α-amino acids (e.g., (Gly)ₙ or (Ser)ₙ), as well as longer chained amino acids, such as hexanoic amino acid; alkylamine (e.g., alkyldiamines); thioalkylamines; thioalkyls; and alkyl chains of C₄, C₅, C₁₂, or longer.

Linkers can have reactive functional groups, which can be protected as required. In some embodiments, the linkers can have homofunctional or heterofunctional reactive groups, including, among others, amine, imidoester, N-hydroxysuccinimide ester, maleimide, aldehyde, carboxyl, haloacetyl, thiol, pyridyldisulfide, hydrazide, carbodiimide, and phosphoramidite groups. In some embodiments, the reactive groups can be a photoreactive group, such as arylazides. Reactive groups on linkers are described in various reference works and publications, such as Hermanson, G. T., 1996, Bioconjugate Techniques, Academic Press, Inc., San Diego, Calif.; Pierce Applications Handbook/Catalog, 2005, Pierce Chemicals; Double-Agents Cross-Linking Guide, 2003, Pierce Biotechnology, Publication No. 1600918; U.S. Pat. No. 6,320,041; Morocho et al., 2004, Bioconjugate Chem 15:569-575; Shchepinov et al., 1997, Nucleic Acids Res 25(6):1155-1161; and Shea et al., 1990, Nucleic Acids Res 18(13):3777-3783. For use in attaching a linker during synthesis of the nucleobase polymers, various linkers with protecting groups can be used, which can be found in Green and Wuts, 1999, Protective Groups in Organic Synthesis, 3^{rd} Ed., Wiley-Interscience).

In some embodiments, the linker can be a cleavable linker that, when subjected to the appropriate conditions, cleaves to separate the molecules connected by the linker. The cleavable linker may be cleavable by a chemical agent, an enzyme, or by photoreaction (see, e.g., Lloyd-Williams et al., 1993, Tetrahedron 49, 11065-11133). Non-limiting examples of chemically cleavable linkers include, among others, vinyl sulphones (WO 00/02895); base-cleavable sites, such as esters (e.g., succinates) cleavable using, for example, ammonia or trimethylamine; acid-cleavable sites, such as benzyl alcohol derivatives, cleavable using trifluoroacetic acid, acetals and thioacetals; dithiols cleavable by thiol compounds (see, e.g., Thevenin et al., 1992, Eur J Biochem 206(2):471-7); sulfonyl compounds cleavable by trifluoromethane sulfonic acid, trifluoroacetic acid, thioanisole; diisopropyldialkoxysilyl linkers cleavable by fluoride ions; and hydrazone linkers (Laguzza et al, 1989, J Med Chem 32:548-555). Other cleavable linkers are described in the literature, for example, Brown, 1997, Contemporary Organic Synthesis 4(3):216-237; W. A. Blattler et al, Biochemistry 24:1517-1524 (1985); Wong, S. S., 1993, Chemistry of Protein Conjugation and Cross-linking, CRC Press, BocaRaton, Fla.; and U.S. Pat. Nos. 4,542,225, 4,569,789, 4,618,492, and 4,764,368. All publications incorporated herein by reference.

In various embodiments, the linkers can be attached to the polypurine tracts through various reactive groups on the nucleobase polymers. Such groups will be apparent to the skilled artisan depending on the nucleobases present and the type of polymer backbone. In some embodiments, the linker can connect the polypurine tracts through one of either a carboxylic group or amine group on each respective tract. In some embodiments, the polypurine tracts can each be connected to the linker through an amine group. In other embodiments, the polypurine tracts can each be connected to the linker through a carboxylic group. In still other embodiments, one of the polypurine tracts can be connected to the linker through an amine group while the other polypurine tract can be connected to the linker through a carboxylic group. In some embodiments, the amine group and the carboxylic group can be functional groups of a polyamide backbone, as described above. It is also to be understood that two linked polypurine tracts can have different orientations with respect to the polypurine strand of the duplex, for example, such as where the polypurine strand has a sugar-phosphate backbone. For example, for a TFNP with a PNA backbone, the two polypurine tracts can both be oriented in the parallel orientation or in the antiparallel orientation. In other embodiments, one of the linked polypurine tracts can be in a parallel orientation while the other linked polypurine tract can be in a antiparallel orientation. It is to be understood from the descriptions herein that the polypurine tracts on the TFNP can be complementary to polypyrimidine tracts on different nucleobase polymer strands of the duplex and is not limited to polypyrimidine sequences on a single nucleobase polymer strand of the duplex.

In some embodiments, the nucleobase polymers forming the triplex can have additional components present at one or both ends of each polymer. In some embodiments, as described above, the components can be additional nucleobase polymer subunits, which can be of any number and type. For example, the nucleobase polymer subunits can be those of polynucleotides, polynucleotide analogs, or polynucleotide mimics. Thus, in some embodiments, the first and/or second nucleobase polymers can be part of a longer polynucleotide, such as, for example, an amplified nucleic acid produced by various amplification reactions, including, among others, polymerase chain reaction or ligase chain reaction. In some embodiments, the first and/or second nucleobase polymers of the triplex can be part of a gene or gene fragment, such as, for example, a DNA fragment of a gene, an RNA transcript, or a cDNA copy of an RNA. In some embodiments, the first and/or second nucleobase polymers of the triplex can be part of a chromosome in which the third nucleobase polymer is annealed to the chromosome, for example, for the purposes of detecting in situ a sequence on a chromosome, either within a cell or in various chromosome preparations (e.g., chromosome spreads, FACS sorted chromosomes, etc.).

In some embodiments, the third nucleobase polymer can comprise one or more linked positively charged groups. It is known that attachment of positively charged groups to PNAs with N-(2-aminoethyl)glycine backbones can stabilize the formation of a triplex based on Hoogsteen/Reverse Hoogsteen hydrogen bonding (see, e.g., Gangamani et al., 1997, Biochem Biophys Res Commun 240(3):778-82; Harrison et al., 1999, Bioorg Med Chem Lett 9(9):1273-8; and Abibi et al., 2004, Biophys J 86(5):3070-8). Without being limited by theory, it is believed that neutralization of negatively charged sugar-phosphate backbones on the duplex segment by positive charges placed onto a TFNP can enhance the stability of the triplex structure. Linked positively charged groups, such as polylysine and polyspermine, are described in Gangamani et al., supra; Harrison et al., supra; WO 03/092736; WO 03/092735; and WO 01/76636, the disclosures of which are incorporated herein by reference.

In some embodiments, the nucleobase polymers of the triplex can be chimeric nucleobase polymers. For example, the TFNP can have a first segment of PNA that hydrogen bonds to the double-stranded portion of a target nucleic acid to form the triplex and also have a second segment comprised of a polynucleotide, such as a deoxyribonucleotide polymer, that anneals to a single-stranded portion of the target nucleotide, thereby forming a stable double-stranded and triple-stranded complex (see, e.g., WO 95/14706). In other exemplary embodiments, the TFNP can have a first nucleobase polymer segment that forms a triplex with a double-stranded target polynucleotide and also have a second nucleobase polymer segment comprised of a polynucleotide, polynucleotide analogs, or polynucleotide mimic useful as a capture sequence or a sequence tag for isolating the triplex composition. It is to be understood that the first and second nucleobase polymers forming the duplex part of the triplex can also be comprised of chimeric nucleobase polymers.

4.3 Labels, Capture Tags, and Substrates

In various embodiments, the triplex can comprise a detectable label. The detectable label can be on any strand of the triplex and can include any type of label for labeling the triplex. In some embodiments, the label can be a direct label, i.e., a label that itself is detectable or produces a detectable signal, or it may be an indirect label, i.e., a label that is detectable or produces a detectable signal in the presence of another compound. The method of detection will depend upon the label used, and will be apparent to those of skill in the art. Examples of suitable direct labels include radioactive, fluorescent, phosphorescent, luminescent, electroluminescent, and electron transfer compounds. Additional detectable labels include Raman labels, nanoparticles, and quantum dots. In some embodiments, the detectable label can be a radioactive label, either incorporated directly into the nucleobase polymer or as part of a tag that is attached to the polymer. Exemplary radiolabels include, by way of example and not limitation, ³H, ¹⁴C, ³²P, ³⁵S, ³⁶Cl, ⁵⁷Co, ¹³¹I and ¹⁸⁶Re. In some embodiments, the detectable label can be comprise a non-radioactive isotope, such as, for example, ²H, ¹⁴C, ¹⁵N, ¹⁸O, etc., that is detectable by suitable spectroscopic techniques (e.g., nuclear magnetic resonance, mass spectroscopy, etc.).

In some embodiments, the label can comprise an enzymatic label that can be detected by conversion of a suitable substrate into a detectable form. Exemplary enzymatic labels for labeling the nucleobase polymers include, among others, alkaline phosphatase, horseradish peroxidase, β-galactosidase, glucourodinase, and glucose oxidase.

In some embodiments, the label comprises a binding moiety, including, among others, biotin, haptens, and peptide tags, which can be bound by corresponding binding partners, such as streptavidin for biotin and antibodies for haptens and peptide tags.

In some embodiments, the detectable label can comprise a chromophore, which refers to a moiety having absorption characteristics, i.e., is capable of excitation upon irradiation by any of a variety of photonic sources. Chromophores can be fluorescing or nonfluorescing, and include, among others, luminescent, chemiluminescent, and electrochemiluminescent molecules and various dyes and fluorophores.

In some embodiments, the chromophore label can comprise a fluorophore. Suitable fluorescent molecules include fluorophores based on xanthene, fluorescein (such as disclosed in U.S. Pat. Nos. 4,318,846 and 6,316,230, and Lee et al., 1989, Cytometry 10:151-164), rhodamine, cyanine, phthalocyanine, squaraine, and bodipy dyes (see, e.g., Molecular Probes Handbook, 10th Ed., R. P Haugland ed., Molecular Probes, Eugene, Oreg. (2005); Smith et al., 1987, Meth Enzymol 155:260-301; and Karger et al., 1991, Nucl Acids Res 19:4955-4962). Exemplary fluorescent dyes include, by way of example and not limitation, 5-carboxyfluorescein (5-FAM), 6-carboxyfluorescein (6-FAM), fluorescein-5-isothiocyanate (FITC), 2′7′-dimethoxy-4′5′-dichloro-6-carboxyfluorescein (JOE); rhodamine and rhodamine derivatives such as N,N,N′,N′-tetramethyl-6-carboxyrhodamine (TAMRA), 6-carboxyrhodamine (R6G), tetramethyl-indocarbocyanine (Cy3), tetramethyl-benzindocarbocyanine (Cy3.5), tetramethyl-indodicarbocyanine (Cy5), tetramethyl-indotricarbocyanine (Cy7), 6-carboxy-X-rhodamine (ROX); hexachloro fluorescein (HEX), tetrachloro fluorescein TET; R-Phycoerythrin, 4-(4′-dimethylaminophenylazo) benzoic acid (DABCYL), and 5-(2′-aminoethyl)aminonaphthalene-1-sulfonic acid (EDANS) (see, e.g., U.S. Pat. Nos. 4,997,928; 4,855,225; and 5,188,934). In some embodiments, suitable fluorescent labels also include fluororescent proteins and fluorescent peptides. Exemplary fluorescent proteins include, but are not limited to, green fluorescent protein (GFP; Chalfie, et al., 1994, Science 263(5148):802-805), EGFP (Clontech Laboratories, Inc., Palo Alto, Calif.), blue fluorescent protein (BFP; Quantum Biotechnologies, Inc. Montreal, Canada; Heim et al, 1996, Curr Biol 6:178-182; Stauber, 1998, Biotechniques 24(3):462-471), enhanced yellow fluorescent protein (EYFP; Clontech Laboratories, Inc., Palo Alto, Calif.), and renilla fluorescent protein (see, e.g., WO 92/15673; WO 95/07463; WO 98/14605; WO 98/26277; WO 99/49019; U.S. Pat. No. 5,292,658; U.S. Pat. No. 5,418,155; U.S. Pat. No. 5,683,888; U.S. Pat. No. 5,741,668; U.S. Pat. No. 5,777,079; U.S. Pat. No. 5,804,387; U.S. Pat. No. 5,874,304; U.S. Pat. No. 5,876,995; and U.S. Pat. No. 5,925,558).

In some embodiments, the chromophore can comprise an acceptor or donor chromophore suitably positioned to permit energy transfer between a complementary donor or acceptor chromophore. In some embodiments, the chromophore donor and acceptor moieties undergo fluorescence resonance energy transfer (FRET), a non-radiative transfer of energy from one fluorophore (the donor) to another (the acceptor). While not being bound by any posited mechanism, upon excitation of the donor, energy transfer to an acceptor molecule results in emission of light at a different wavelength. FRET is not mediated by photon emission and does not require the acceptor to be fluorescent, although many applications of FRET use fluorescent donor and acceptor molecules. If the acceptor is also fluorescent, the transferred energy can be emitted as fluorescence characteristic of the acceptor. If the acceptor is not fluorescent, the energy can be lost through equilibration with solvent. Energy transfer can result in quenching of donor fluorescence, a reduction of the fluorescence lifetime, and a corresponding increase in acceptor fluorescence emission. When the donor and acceptors are different, FRET can be detected by the appearance of acceptor fluorescence or by quenching of donor fluorescence. When the donor and acceptor are the same, FRET can be detected by the resulting fluorescence depolarization. FRET is strongly dependent on the distance separating the donor and acceptor and their orientation to one another. Consequently, FRET can also provide information on the distance and conformation between a donor and acceptor. Since a third nucleobase polymer and the nucleobase polymers of the duplex are associated together in the triplex, in some embodiments, FRET interactions are possible if one of the donor/acceptor pair is present on the third nucleobase polymer and the other member of the pair is present on at least one of the nucleobase polymers of the duplex and the donor/acceptor pair is in sufficient proximity to permit energy transfer. Suitable donor/acceptor pairs will be apparent to the skilled artisan. Exemplary FRET pairs include, by way of example and not limitation, fluorescein and rhodamine, Cy5 and tetramethyrhodamine, Cy3 and Cy5, Cy3B and Cy5, Cy5 and Cy7Q, tryptophan and danysl, BODIPY FL and BODIPY FL, rhodamine and malachite green, phycoerythrin and Cy5, dansyl and octadecylrhodamine, perylene (Pe) and terrylenediimide (TDI), and green fluorescent protein (from Aequoria Victoria) and yellow fluorescent protein (YFP) (see, e.g., Wu and Brand, 1994, Anal Biochem 218:1-13). Donor acceptor pairs with high quantum efficiency and large Stokes shift between absorption spectra of the donor and emission spectra of the acceptor is desirable. The Forster distance, which is the separation distance where the probability of the energy transfer is 50%, are known to the skilled artisan or can be determined for any donor/acceptor pair.

In some embodiments, the donor and acceptor chromophores are selected for fluorescence quenching, which refers to any process that decreases the fluorescence intensity of a given substance. While not being bound by any mechanistic explanations for quenching, a variety of processes can result in quenching, such as excited state reactions, energy transfer, complex formation, and/or collisional quenching (see, e.g., Yaron et al., 1979, Anal Biochem 95:228-235). Fluorescence quenching can arise in the context of two different fluorescent molecules, such as quenching observed in FRET described above, and in the context of two of the same fluorescent molecules (i.e., self-quenching). Fluorescence quenching can also occur in the context of a non-fluorescent dye molecule that absorbs the energy of the excited fluorescent molecule, such as by energy transfer or collisional quenching. Without intending to be bound to any theory, in some contexts, quenching is believed to occur through a combination of quenching processes, for example, a combination of FRET and non-FRET quenching (see, e.g., U.S. Pat. No. 6,485,901). Exemplary non-fluorescent quenchers useful for detection by quenching, include as non-limiting examples, dabcyl, Black Hole Quenchers® (e.g., BHQ®-0 BHQ®-1, BHQ®-2, and BHQ®-3), and QST dyes (e.g., QSY-7, QSY-9, QSY-21, and QSY-35). Others are described in WO 03/019145 and U.S. Pat. Nos. 6,790,945; 6,727,356; 6,790,945; and Clegg, 1992, Methods in Enzymology 211:353-389, the disclosures of which are incorporated herein by reference.

In various embodiments, the donor and acceptor chromophores are suitably positioned to allow energy transfer to detect proximity of the donor and acceptor chromophores and hence the proximity of the moieties attached to the chromophores, such as the nucleobase polymers forming the triplexes. In various embodiments, the linkers and spacers used for connecting the polypurine tracts, as described above, are also suitable for attaching the chromophores to the nucleobase polymers such that formation of the triplexes positions the chromophores for proper energy transfer. Determining suitable distances between chromophores for various applications herein are well within the skill of those in the art (see, e.g., U.S. Pat. Nos. 5,565,322, 6,485,901, and 6,787,304).

In some embodiments of the triplexes herein, the third nucleobase polymer can comprise a donor or acceptor chromophore, and the first and/or second nucleobase polymer comprises a corresponding donor or acceptor chromophore to form a donor-acceptor chromophore pair with the chromophore on the third nucleobase polymer. The donor and acceptor chromophore pair is suitably positioned such that, in the assembled triplex, energy transfer is possible between the donor and acceptor chromophores. In some embodiments, the chromophore on the third nuclease base polymer is a FRET acceptor. In other embodiments, the chromophore on the third nucleobase polymer is a FRET donor.

In some embodiments, the chromophore of the third nucleobase polymer is one of either a fluorescence quencher or fluorophore and the chromophore of the first and/or second nucleobase polymer is the other one of either a fluorescence quencher or a fluorophore. In some embodiments, the third nucleobase polymer can have both a donor and acceptor chromophore (see, e.g., WO 99/21881; and U.S. Pat. Nos. 6,355,421, 6,485,901, and 6,649,349). In some embodiments, the third nucleobase polymer comprising both a donor and acceptor chromophore can further comprise an intercalator.

In some embodiments, the label is a mobility modifier. “Mobility modifier” refers to a moiety capable of producing a particular mobility in a mobility-dependent analysis technique, such as, electrophoresis (see, e.g., U.S. Pat. Nos. 5,470,705, 5,514,543, 6,395,486 and 6,734,296). Thus, in some embodiments, a mobility modifier is an electrophoresis mobility modifier. In some embodiments, an electrophoresis mobility modifier can be a polynucleotide polymer or a non-polynucleotide polymer. Various non-limiting examples of non-polynucleotide electrophoresis mobility modifiers include, but are not limited to, polyethylene oxide, polyglycolic acid, polylactic acid, polypeptide, oligosaccharide, polyurethane, polyamide, polysulfonamide, polysulfoxide, polyphosphonate, and block copolymers thereof, including polymers composed of units of multiple subunits linked by charged or uncharged linking groups.

The use of detectable labels in the detection of triplexes are well within the abilities of the skilled artisan. Factors to be considered in selecting the number and types of detectable labels and their distribution among the various nucleobase polymers, include but are not limited to, the number of target polynucleotides to be analyzed (e.g., single-plex vs. multiplex analysis), the method selected for detecting the modified products of the detection target polynucleotides, the number and types of detectable labels than may be discriminated, and the extent to which each specific target polynucleotide is to be discriminated.

In some embodiments, one or more intercalators are attached to one or more of the nucleobase polymers forming the triplex. “Intercalator” refers to a molecule or chemical group that inserts between two other molecules or groups to form complexes of two or more molecules held together in unique structural relationship other than by covalent bonds, such as by hydrogen bonding, ion pairing, Van der Waals forces, and combinations thereof. In some embodiments, intercalators capable of inserting into double-stranded nucleic acids can be attached to the TFNP to modulate binding of the polymer to the duplex, for example to stabilize the hydrogen bonded triplex forming polymer. Exemplary intercalators include, among others, polycyclic aromatic compounds (e.g., anthracene and pyrene), acridine derivatives (e.g., 9-aminoacridine), anthracycline derivatives (e.g., daunomycin) and anthraquinone derivatives (see, e.g., WO 01/44190). Other intercalators will be apparent to the skilled artisan (see, e.g., Saenger, W., 1984, Principles of Nucleic Acid Structure, Springer-Verlag, New York, N.Y.).

The intercalator can be attached to any part of the TFNP, with or without use of linkers. In some embodiments, the intercalators can replace a nucleobase residue on the nucleobase polymer while in other embodiments, the intercalator can be attached to the nucleobase. In some embodiments, a nucleobase polymer can have a plurality of intercalators. An exemplary embodiment of a TFNP with a plurality of intercalators is one in which at least one intercalator replaces a nucleobase and another intercalator is attached to the nucleobase. Intercalators can also be attached to the backbone, such as, for example, the carboxy or amino functional groups on a nucleobase polymer of PNAs. The attachment can be on the internal residues or the ends of the polymer. Accordingly, in some embodiments, the intercalators can be attached to the triplex of the structure of formula (I) through at least one of the R′ adjacent to the left or right N. In other embodiments, intercalator can be attached to the left R′ residue adjacent to the left N and to the right R′ adjacent to the right N of structural formula (I).

In some embodiments, the intercalator is attached to the TFNP through a linker. The linker can be on a nucleobase or a part of the polymer backbone. Any number of linkers described herein, such as those used for connecting the polypurine tracts to each other, can be adapted for attaching the intercalating agent to the nucleobase polymer.

In some embodiments, the triplex composition can comprise a capture tag. A “capture tag” refers to a member of a binding pair that, when attached to another molecule herein, e.g., a nucleotide, oligonucleotide, nucleobase polymer, a target polynucleotide, can be isolated (e.g., by capture) through interaction with the other member of the binding pair. A capture tag may have one or more tags, which when a plurality of tags are used can be the same or different. Exemplary capture tags include, among others, biotin, which can be incorporated into nucleic acids (Langer et al., 1981, Proc Natl Acad Sci USA 78:6633) and captured using streptavidin or biotin-specific antibodies; a hapten such as digoxigenin or dinitrophenol (Kerkhof, 1992, Anal Biochem 205:359-364), which can be captured using a corresponding antibody; and a fluorophore to which antibodies can be generated (e.g., Lucifer yellow, fluorosceine, etc.). In some embodiments, the capture tag can comprise a specific sequence, referred to as a “capture sequence,” which can be captured using a “capture probe” having a sequence complementary to the capture sequence. These sequences can be attached to the TFNP or attached to one or both of the nucleobase polymers forming the duplex to which the TFNP binds. In other embodiments, the capture tag can be a peptide tag that interacts with its corresponding antibodies or other binding partners. Exemplary peptide tags include, among others, FLAG tag, c-myc tag, polyarginine tag, poly-His, HAT tag, calmodulin binding peptide, and S-fragment of RNase A (see, e.g., Terpe, K, 2003, Appl Microbiol Biotechnol 60:523-533). Other tags for use in labeling the nucleobase polymers will be apparent to the skilled artisan.

In some embodiments, any of the nucleobase polymers can have one or more of the same or different tags. In some embodiments, the tag is attached to the third nucleobase polymer, which permits isolation or detection of the triplex composition containing the third nucleobase polymer. In some embodiments, the tag can be attached to the first and/or second nucleobase polymers. In some embodiments, tags can be present on the third nucleobase polymer and one or both first and second nucleobase polymers. In some embodiments, use of different tags on the third nucleobase polymer and the first and/or second nucleobase polymer allows codetection of the TFNP and one or both first and second nucleobase polymers.

In some embodiments, the nucleobase polymers can be attached to a substrate, as defined and described above. Substrate can be of any material to which nucleobase polymers can be attached. Nucleobase polymers can be attached to the substrate by any chemical or physical means such as through ionic, covalent or other forces well known in the art (see, e.g., Dattagupta et al., 1989, Anal Biochem 177:85-89; Saiki et al., 1989, Proc Natl Acad Sci USA 86:6230-6234; and Gravitt et al., 1998, J Clin Micro 36:3020-3027). If the attachment is covalent, the surface of the substrate can contain reactive groups for attaching the nucleobase polymers to the substrate, including, among others, carboxyl, amino, hydroxyl, and thiol groups.

In some embodiments, nucleobase polymers can be attached to a substrate by means of a linker or spacer molecule, such as that described in U.S. Pat. No. 5,556,752 and described herein. In some embodiments, a linker or spacer molecule can comprise between 6-50 atoms in length and includes a surface attaching portion that attaches to the substrate. Exemplary methods for attachment include, among others, substrates having (poly)trifluorochloroethylene surfaces, or by siloxane bonds (using, for example, glass or silicon oxide as the substrate). Siloxane bonding can be formed by reacting the support with trichlorosilyl or trialkoxysilyl groups of the spacer Aminoalkylsilanes and hydroxyalkylsilanes, bis(2-hydroxyethyl)-aminopropyltriethoxysilane, 2-hydroxyethylaminopropyltriethoxysilane, aminopropyltriethoxysilane or hydroxypropyltriethoxysilane are surface attaching groups. The spacer can also include an extended portion or longer chain portion that is attached to the surface attaching portion of the probe. For example, amines, hydroxyl, thiol, and carboxyl groups are suitable for attaching the extended portion of the spacer to the surface attaching portion. The extended portion of the spacer can be any of a variety of molecules that are inert to any subsequent conditions for polymer synthesis. In some embodiments, these longer chain portions can be aryl acetylene, ethylene glycol oligomers containing 2-14 monomer units, diamines, diacids, amino acids, peptides, or combinations thereof, as discussed above. In some embodiments, the extended portion of the spacer can be a polynucleotide or the entire spacer can be a polynucleotide. The extended portion of the spacer also can be polyethyleneglycols, polynucleotides, alkylene, polyalcohol, polyester, polyamine, polyphosphodiester and combinations thereof. Additionally, for use in synthesis of probes, the spacer can have a protecting group, attached to a functional group, e.g., hydroxyl, amino or carboxylic acid) on the distal or terminal end of the spacer (opposite the substrate). After deprotection and coupling, the distal end can be covalently bound to a nucleobase polymer or probe.

In some embodiments, the nucleobase polymers can be attached to a substrate to generate arrays, including microarrays and high-density arrays. Microarray chips containing a library of probes can be prepared by a number of well known techniques including, for example, light-directed methods, such as described in U.S. Pat. Nos. 5,143,854, 5,384,261 and 5,561,071; bead based methods, such as described in U.S. Pat. No. 5,541,061; and pin based methods, such as described in U.S. Pat. Nos. 5,288,514, 5,556,752, and 6,475,721.

In some embodiments, spotting methods also can be used to prepare a microarray chip with nucleobase polymers immobilized thereon. Reactants are delivered by directly depositing relatively small quantities in selected regions of the support. In some embodiments, the entire support surface can be sprayed or otherwise coated with a particular solution. Typical spotting devices include a micropipette, nanopippette, ink-jet type cartridge or pin to deliver the nucleobase polymer containing solution or other fluid to the support and, optionally, a robotic system to control the position of these delivery devices with respect to the support. Spotting methods are described in, for example, U.S. Pat. Nos. 5,288,514, 5,312,233 and 6,024,138. In some embodiments, the substrate can include a series of tubes or multiple well trays, or a manifold for placing the nucleobase polymers on the substrate surface.

In some embodiments using a substrate, the first and/or second nucleobase polymers forming the duplex segment can be attached. For example, one nucleobase polymer can be attached to the substrate and the duplex segment formed by annealing the other nucleobase polymer to the attached polymer, thereby forming a duplex with the duplex segment of polypurine and polypyrimidine on the substrate. The polypurine tract on the third nucleobase polymer can then be reacted with the duplex on the substrate to generate the triplex structure on the substrate surface.

In other embodiments, the third nucleobase polymer with the polypurine tract forming the triplex can be attached to the substrate. To form a triplex on the substrate, a duplex of a polypurine and polypyrimidine can be reacted with the attached third nucleobase polymer to form the triplex. In various embodiments of this format, the formation of triplex structures can be used to detect the presence of the double-stranded target nucleic acid. In contrast to typical array probes based on hybridization of single-stranded target nucleic acids to single-stranded probes, which can require denaturation of the sample, the use of TFNPs can be used to identify a double-stranded target nucleic acid without the requirement for denaturation.

4.4 Synthesis of Nucleobase Polymers and Triplex Structures

The nucleobase polymers for forming the compositions of triplexes can be made by standard methodologies known in the art. For example, the nucleobase polymers can be synthesized in whole or in parts, where the parts are subsequently joined together.

In some embodiments, nucleobase polymers can be synthesized using standard chemistries (see, e.g., Current Protocols in Nucleic Acid Chemistry, John Wiley & Sons, 2003; U.S. Pat. No. 4,973,679; Beaucage, 1992, Tetrahedron 48:2223-2311; U.S. Pat. No. 4,415,732; U.S. Pat. No. 4,458,066; U.S. Pat. No. 5,047,524 and U.S. Pat. No. 5,262,530; all of which are incorporated herein by reference). Standard synthetic routes for generating nucleobase polymer includes, among others, phosphoramidite, phosphate, and triester chemistries. The synthesis can be accomplished using automated synthesizers available commercially, for example the Model 392, 394, 3948 and/or 3900 DNA/RNA synthesizers available from Applied Biosystems, Foster City, Calif.

Methods for synthesizing polynucleotide analogs and mimetics will also follow standard methodologies. For example, PNAs are described in U.S. Pat. Nos. 5,539,082; 5,527,675; 5,623,049; 5,714,331; 5,718,262; 5,736,336; 5,773,571; 5,766,855; 5,786,461; 5,837,459; 5,891,625; 5,972,610; 5,986,053; 6,107,470; 6,201,103; 6,350,853; 6,357,163; 6,395,474; 6,414,112; 6,441,130; and 6,451,968; all of which are herein incorporated by reference. General descriptions for PNA synthesis methodologies are given in Nielsen et al., 1999, Peptide Nucleic Acids; Protocols and Applications, Horizon Scientific Press, Norfolk England. Synthesis of PNA polymers with extended backbones are also described in Nielsen et al., 1994, Bioconjug Chem 5:3-7.

In other embodiments, recombinant techniques can also be used to synthesize nucleobase polymers of polynucleotides, or parts thereof (see, e.g., Sambrook et al., Molecule Cloning: A Laboratory Manual, 3rd Ed., Cold Spring Harbor Laboratory Press (2001); and Current Protocols in Molecular Biology, Ausubel. F. ed., Greene Publishing Associates (1998), updates to 2005). For example, single-stranded polynucleotides are readily made using single-stranded phage systems. Cloned fragments of naturally occurring sequences as well as amplification products can also be used for constructing the triplex structures.

Where the nucleobase polymer is a composite of non-nucleobase and nucleobase polymers, the polymers can be synthesized in segments and then assembled together or, alternatively, formed by sequential synthesis of the non-polynucleotide polymer region and the polynucleotide polymer region. Non-limiting examples of non-nucleobase polymers include polyethylene glycol (PEG), polystyrenes, polyacrylic acids, polyacetamides, polyphosphates, and other polymers that do not form Watson and Crick or Hoogsteen base-pairs with a nucleobase polymer. The synthetic polymers can be block polymers or block copolymers. An exemplary composite nucleobase polymer is a polymer formed with a block polymer of polyethylene glycol and a polymer of deoxypolynucleotides, as described in Sanchez-Quesada et al., 2004, Angew Chem Int Ed 43:3063-3067 and Jaschke et al., 1994, Nucleic Acids Res 22(22):4810-4817. Other composite polymers of polynucleotides and non-polynucleotide polymers or linkers are described in, among others, U.S. Patent Application No. 2005/0153926; Greenberg et al., J Org Chem 66:7151-7154; and Pon and Yu, 2005, Nucleic Acids Res 33(6):1940-1948; the disclosures of which are incorporated herein by reference.

The triplex structures can be made in any number of ways. In some embodiments, the method of forming the triplex can comprise annealing a first nucleobase polymer comprising a polypurine tract to a second nucleobase polymer comprising a polyprimidine tract, wherein the polypyrimidine tract is complementary to the polypurine tract of the first nucleobase polymer, thereby forming a duplex comprising a duplex segment of polypurine and polypyrimidine (e.g., poly(purine:pyrimidine)). The duplex is then contacted with a TFNP comprising a second polypurine tract complementary to the polypyrimidine tract of the duplex segment under conditions suitable for each purine base of the second polypurine tract to hydrogen bond to both purine and pyrimidine bases of a purine:pyrimidine base-pair of the duplex segment.

In some embodiments, all of the nucleobase polymers capable of forming the triplex structure are contacted together without first forming a duplex. Because of its extended backbone, the TFNP is not expected to form stable complexes with either of the nucleobase polymers used to form the duplex. Consequently, the duplex with the duplex segment of annealed polypurine and polypyrimidine can form spontaneously in the presence of the TFNP, which can then hydrogen bond to the formed duplex to generate the triplex.

The conditions for forming the triplex structures can be determined by those skilled in the art and will take into account factors typically affecting formation of duplex and triplex nucleic acids. These factors include, among others, temperature, salt concentration (i.e., ionic strength), cation concentration, pH, detergent concentration, presence or absence of chaotropes, nucleobase polymer sequence, and length of duplex segment bound by the TFNP. Suitable conditions for forming a triplex structure can be found by the well-known technique of fixing several of the aforementioned factors and then determining the effect of varying a single factor. Guidance is provided in various reference works, such as Sambrook et al., Molecule Cloning: A Laboratory Manual, 3rd Ed., Cold Spring Harbor Laboratory Press (2001), Current Protocols in Molecular Biology, Ausubel. F. ed., Greene Publishing Associates (1998), updates to 2005. Detecting the formation of triplex structures can be done using any number of methods disclosed herein.

4.5 Assays for Triplex Formation

A variety of techniques can be used to detect the formation of triplex structures. In some embodiments, the detection can be based on melting transitions of the hydrogen bonded nucleobase polymers in the triple-stranded structures. Determining melting profiles can use spectroscopic changes caused by dissociation of the nucleobase polymers as a function of temperature (see, e.g., Plum et al., 1990, Proc Natl Acad Sci USA 87(23): 9436-9440; Xodo et al., 1990, Nucleic Acids Res. 18(12): 3557-3564). Dissociation/association of nucleobase polymers in the triplex can be measured by various techniques, such as by UV absorbance or circular dichroism. calorimetric techniques for detection of triplex structures can use differential scanning calorimetry in which the difference in the amount of heat required to increase the temperature of a sample and reference are measured as a function of temperature. Examples of differential calorimetric measurements of triplex structures are described in Plum et al., 1995, J Mol. Biol. 248(3):679-95; He et al., 1997, Biopolymers 41(4):431-41; and Sugimoto et al., 2001, Biochemistry 40(31):9396-405; the disclosures of which are incorporated herein by reference.

In some embodiments, the triplex structures can be detected based on differences in electrophoretic mobility caused by annealing of a third nucleobase polymer to the duplex (see, e.g., Catapano et al., 2000, Biochemistry 39:5126-5138). Typically, a nucleobase polymer of the duplex or the TFNP is labeled with a detectable label, for example a radioactive or fluorescent label. Compositions of triplexes can be formed by mixing the nucleobase polymers and then separating the products by electrophoresis through an electrophoresis medium, such as, for example, crosslinked polyacrylamide. Labeled products separated by electrophoresis can be detected by detecting the presence of the label. Differential mobilities of single-stranded, double-stranded, and triple-stranded structures in the electrophoretic medium allow for the identification of the triplex structure from the single-stranded and double-stranded forms (McGuffie et al., 2002, Nucleic Acids Res 30(12):2701-2709).

In some embodiments, the formation of triplex structures can be detected using FRET or fluorescence quenching between donor chromophores and acceptor chromophores, as described above. In some embodiments, one or more donor or one or more acceptor chromophores can be attached to the TFNP. A corresponding acceptor or donor chromophore is attached to the first or second nucleobase polymers and suitably positioned to permit interaction between the donor-acceptor chromophores upon formation of the triplex. Such methods for detecting triplex formation are described in various references, for example, Reither et al., 2002, BMC Biochem 12:3(1):27; and Yang et al., 1994, Biochemistry 33(51):15329-37, the disclosures of which are incorporated herein by reference.

In some embodiments, the assay for formation of triplex structures can use electron microscopy or atomic force microscopy. Detection of the triplex structures by electron microscopy can use a tag, such as biotin, attached to the third nuclease polymer forming the triplex structure, which is then made visible by binding a label detectable by electron microscopy (see, e.g., Demidov et al., 1994, Nucleic Acids Res. 22:5218-5222). For example, when biotin is the tag, streptavidin label allows visualization of the label by shadowing with metal vapor. Other electron dense particles, such as gold particles or deposition of an electron dense chemical by streptavidin enzyme conjugates, can also be used. Either conventional electron transmission microscopy or scanning electron microscopy can be used for visualization (Chemey et al., 1998, Biophysical J74:1015-1023). Thickness of strands can be obtained from image measurements of nucleic acid molecules.

Similarly, formation of triplex structures can be determined by scanning probe microscopy techniques. Samples can be prepared similar to techniques used for electron microscopic detection; although a triplex structure is identifiable without resort to use of labels (Hansma et al., 1996, Nucleic Acids Res. 24:713-720). Atomic force measures the surface contours of a molecule by placing a cantilever with a sharp tip, typically made of silicon or silicon nitride, in close proximity to a sample surface. Deflection of the cantilever caused by van der Waals forces between the tip and the molecule can be measured. The triplex can be immobilized on a flat surface, such as mica, to minimize artifactual surface differences (Vasenka et al., 1992, Ultramicroscopy 42-44 (Pt B):1243-9).

In some embodiments, the triplex structures can be detected by nuclear magnetic resonance (NMR) spectroscopy. Detection using NMR can involve both one dimensional, two dimensional, and three dimensional NMR (see, e.g., Wang et al., 1992, Biochemistry 31:4838-4846; and Jiang et al., 2001, Nucleic Acids Res 29(20):4231-4237). Use of Nuclear Overhauser effect techniques, such as in NOESY, COSY and TOCSY-NOESY, can be used to refine structure determination by NMR (see, e.g., Sørensen et al., 2004, Nucleic Acids Res 32(20):6078-6085).

Other methods of detecting the formation of triplex structures can include, among others, nucleic acid structure probes (see, e.g., Collier et al., 1991, Nucleic Acids Res 19(15):4219-24), sedimentation techniques, chromatography, and antibodies to nucleobase polymers. Other methods will be apparent to the skilled artisan.

4.6 Uses of the Triplex Structures

The triplex structures and methods of its synthesis are useful in a variety of applications, such as in diagnostic, pharmacological, and research methods.

In some embodiments, the TFNP can be used to purify nucleic acids. In various embodiments, the third nucleobase polymer forming the triplex can have a capture tag or attached to an isolatable substrate. Because the triplex forming polymer binds to double-stranded polynucleotides, the TFNP can be contacted with a sample containing the target duplex polynucleotides. Use of capture tag or substrate, such as a bead, allows the triplex complex to be readily isolated. Choice of appropriate sequences for the TFNP can allow a specific double-stranded target polynucleotide to be purified away from other double-stranded polynucleotides.

In some embodiments, the TFNP can be used to detect the presence, absence, and/or quantity of target polynucleotides in a sample (see, e.g., WO 00/05408). In various embodiments, at least one analyte comprises at least one target sequence, which is a nucleobase sequence, including but not limited, to at least one genomic DNA (gDNA), RNA (e.g., mRNA; noncoding RNA, tRNA, siRNA, snRNA), nucleic acid obtained from subcellular organelles (e.g., mitochondria or chloroplasts), and nucleic acid obtained from microorganisms, parasites, or viruses. The target nucleic acid to be detected can be present in double-stranded form, although target nucleic acids in single-stranded form can be readily converted to a double-stranded form by annealing a complementary strand or by replicating the complementary strand by known procedures. Discussions of target acids can be found in, among others, Current Protocols in Nucleic Acid Chemistry, S. Beaucage, D. Bergstrom, G. Glick, and R. Jones, eds., John Wiley & Sons (1999) including updates through August 2005.; S. Verma et al., 1998, Ann Rev Biochem 67:99-134; and Eddy, S., 2001, Nature Rev Genetics 2:919-29.

In some embodiments, the target nucleic acid can be associated with a sequence variation within a population. These sequence variations can be used in, for example, evolutionary studies, determining family relationships, forensic analysis, disease diagnosis, disease prognosis, and disease risk assessment. In some embodiments, the target polynucleotide can be a single nucleotide polymorphism or SNP. In some embodiments, the target polynucleotide can be associated with genetic abnormality, including somatic and heritable mutations, non-limiting examples of which are nonsense mutations, missense mutations, insertions, deletions, and chromosomal translocations.

In some embodiments, the target nucleic acid of interest can be an amplicon generated by any suitable amplification technique including, but not limited to PCR, OLA, LCR. RCA, and RT-PCR (see, e.g., U.S. Pat. Nos. 4,683,195; 4,683,202; 4,800,159; 4,965,188; 5,075,216; 5,130,238; 5,176,995; 5,185,243; 5,354,668; 5,386,022; 5,427,930; 5,455,166; 5,516,663; 5,656,493; 5,679,524; 5,686,272; 5,869,252 6,025,139; 6,040,166; 6,197,563; 6,297,016; 6,514,736; and European Patent Nos. EP-A-0200362, EP-A-0201184, and EP-A-320308). Amplicons suitable for use in the methods herein can be obtained from cells, cell lysates, and tissue lysates. In some embodiments, the primers used for the amplification reaction can have, in addition to the specific sequence for annealing to the target amplicon, a detection sequence of polypurine or polypyrimidine that when replicated into double-stranded form bind the TFNP in a sequence specific manner.

The samples to be analyzed can be obtained from various sources. “Sample” is to be used in the broad sense and is intended to include a wide range of environmental sources and biological materials, including compositions derived or extracted from such biological materials. Non-limiting examples of environmental samples include food, water, soil, waste, or air. Exemplary biological samples include, among others, whole blood; red blood cells; white blood cells; buffy coat; hair; nails and cuticle material; swabs (e.g., buccal swabs, throat swabs, vaginal swabs, urethral swabs, cervical swabs, throat swabs, rectal swabs, lesion swabs, abcess swabs, nasopharyngeal swabs, and the like); urine; sputum; saliva; semen; lymphatic fluid; amniotic fluid; cerebrospinal fluid; peritoneal effusions; pleural effusions; fluid from cysts; synovial fluid; vitreous humor; aqueous humor; bursa fluid; eye washes; eye aspirates; plasma; serum; pulmonary lavage; lung aspirates; and tissues, including but not limited to, liver, spleen, kidney, lung, intestine, brain, heart, muscle, pancreas, biopsy material, and the like. Tissue culture cells, including explanted material, primary cells, secondary cell lines, and the like, as well as lysates, extracts, or materials obtained from any cells, are also within the meaning of the term biological sample as used herein. Microorganisms and viruses that can be present on or in a sample are also within the scope of the invention. Materials obtained from forensic settings are also within the intended scope of the term sample.

In some embodiments, the target polynucleotide can comprise a gene or chromosome in a cell. In these embodiments, the TFNPs can be used to detect specific sequences directly in cells without processing the cells under denaturing conditions since the TFNP bind directly to double-stranded nucleic acids. By using a labeled TFNP, in situ detection of specific sequences in a manner similar to fluorescence in situ hybridization (FISH) can be done for genetic analysis of chromosomes, fluorescence activating cell sorting, and detection of pathogens.

In some embodiments, the TFNP can be used to detect target polynucleotides in an array and microarray format, as described above, or in various biosensors, such as BIAcore sensors. Without limitation and for purposes of illustration, an exemplary microarray format is a chip in which a TFNP comprising a polypurine tract is attached to the chip surface. The TFNP capable of binding a duplex target polynucleotide in a sequence specific manner can be labeled with a fluorescent label. A sample comprising a population of amplified duplex polynucleotides can be contacted onto the chip under conditions suitable for formation of triplex structures. Duplex polynucleotides with a poly(purine:pyrimidine) sequence complementary to the polypurine sequence of the TNFP can become bound to labeled TNFP, which can allow detection of the fluorescent label. TFNPs with different sequences can be used to detect different target duplex polynucleotides in a sample.

In some embodiments, the TFNPs can be used to target delivery of drugs and other compounds to a specific polynucleotide sequence of interest. A variety of compounds can be delivered to target sequences of interest.

In some embodiments, TFNPs can also be used for modulating biological process involving duplex nucleic acid structures. In some embodiments, the triplexes of the present disclosure can act as a structural obstacle to block function of RNA polymerase, thereby modulating transcription of both prokaryotic and eukaryotic origins. Alternatively, the binding of the TFNPs disclosed herein in the major groove of duplex polynucleotide can prevent access or interaction of the transcription machinery with the DNA. Regardless of the mechanism of modulation, duplex segments of polypurine and complementary polypyrimidine can be identified in regions important for transcription activity (e.g., RNA polymerase interaction, transcription factor binding, and enhancer binding sequences) and TFNPs with appropriate sequence complementarity used to form triplex structures for modulating transcription. In this way, TFNPs can be used to target genes involved in cellular processes, including gene involved in various disease conditions. Other biological processes that can be modulated include, among others, DNA replication and recombination mechanisms.

4.7 Kits

The present disclosure further provides kits comprising components for generating the triplex structures, including, the first nucleobase polymer and/or second nucleobase polymer for forming the duplex, and/or the TFNP with the polypurine tracts that hydrogen bond to the duplex to form the triplex structures. The nucleobase polymer can be unlabeled or labeled with a detectable label or a capture tag. In some embodiments, the kit contains substrates (e.g., beads or microarrays) to which nucleobase polymers can be attached for detecting target polynucleotides.

The kits can also contain reagents for forming the triplex structures, as well as reagents for detecting the structures formed. These include buffers, salts, divalent ions, nucleic acid dyes, and additives, including those that can aid in stabilizing the triplex structures. The kit can also comprise instructions in various mediums (e.g., compact disc, video tape, printed form, memory cards, etc.) for teaching and guiding the practitioner in the proper use of the kit components and analysis of the results.

### 5. EXAMPLES

Aspects of the present teachings may be further understood in light of the following examples, which should not be construed as limiting the scope of the present teachings in any way.

### 5.1 Example 1

### Molecular Modeling of Triplex Structures

Molecular modeling studies were carried out by identifying hydrogen bonding information in the major groove of a Watson-Crick base-paired double-stranded deoxyribonucleic acid. After identifying possible hydrogen bonding schemes, a space-filling model of Watson-Crick base-paired polynucleotide was built using a variety of PNA backbones with adenine and guanine attached at major groove sites that correspond to AT and GC pairs respectively. Simple model building on extended platforms to support the additional purines, bonded with “Straus-Matysiak” hydrogen bonding in the major groove, showed that the standard PNA backbone was too short to permit the addition of a third strand in the major groove. However, the extended PNA backbones described above ((N-(2-aminoethyl)-β-alanine backbone and N-(3-aminopropyl)-glycine backbone), permitted the construction of a third strand for 16 nucleobases without any detectable strain on the backbone because of backbone length constraints. A ball and stick model is illustrated in FIG. 6A in which an anti-parallel strand PNA third strand winds from bottom to top in the major groove of Watson-Crick paired double stranded DNA. A space filling model of a triplex with anti-parallel (bottom portion) and parallel (top portion) third strand of a PNA is shown in FIGS. 6B-6D. The PNAs in the illustrated models have an N-(2-aminoethyl)-β-alanine backbone.

### 5.2 Example 2

### Synthesis of PNAs with Extended Backbones

Synthesis of PNA polymers using N-(3-aminopropyl)glycine monomers, denoted herein as “apg”, incorporates the Boc/Z protecting group strategy, in which t-Boc and benzyloxycarbonyl groups mask the N-terminal and exocyclic amines, respectively, as shown below. All apg-monomers were prepared by Niels Clauson-Kaas A/S, Denmark.

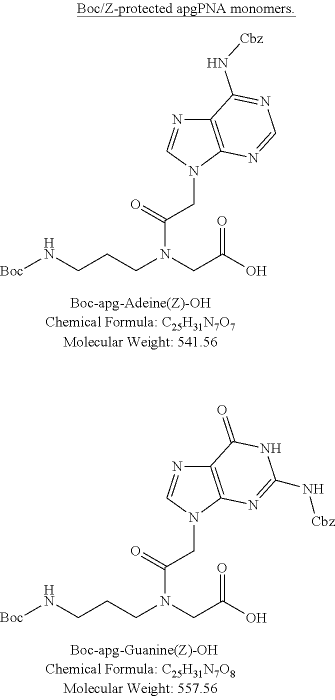

### Synthesis

All apgPNA polymers were prepared on an ABI 433A using Boc chemistry. The synthesis protocols were based on previously published methods, such as that described in Koch et al., 1997, “Improvements in automated PNA synthesis using Boc/Z monomers,” J Pept Res 49:80-8)

Generally, the apgPNA oligomers were synthesized at either a 5 or 10 μmol scale, using a previously prepared Boc-Lysine(Fmoc)-MBHA resin (˜50 or 100 mg at a ˜0.1 μmol/mg loading). During coupling, 6.5 equivalents of monomer (32.4 or 64.8 μmol at 0.18 M in NMP) were preactivated with HATU as activator (0.19 M in DMF) and DIEA as base (0.4 M in DMF). The coupling was repeated a second time (double-coupling) during all syntheses, followed by a capping step with acetic anhydride in DMF.

After synthesis of a base 10′ mer sequence was completed, the resin was washed with DCM, vacuum dried, and weighed. The resin was split into two portions, of which approximately ⅓ was retained as the 10′ mer, and the remaining ⅔ was used for synthesis of the 11′ mer. Following completion of the 11′ mer (addition of one monomer to the 10′ mer sequence), a similar process was followed. In this instance, the 11′ mer resin was split into two portions, one half of which was used for the 12′ mer synthesis.

Prior to cleavage, the C-terminal lysine Fmoc group was removed using 20% piperidine in DMF, the resin washed with DMF and DCM (each 3×3 mL) and neutralized/acidified with 20% TFA in DCM. Cleavage and final deprotection of the apgPNAs were effected with a cleavage cocktail (7:2:1, TFA:TFMSA:m-cresol). Approximately 750 μL of the cocktail was added to each resin and allowed to sit at room temperature for 2 hours. The cleaved apgPNAs were precipitated and washed with diethyl ether (4×1 mL) and dried on a 50° C. heat block.

Crude Analysis and Purification

The dried, cleaved apgPNAs were reconstituted in 750 μL of 0.1% TFA/25% ACN (aq). Analysis of the crude product was carried out by MS, HPLC, and OD quantification. A 1000 fold dilution of the crude samples was used for the OD measurement. 1:10 and 1:100 dilutions in sinapinic acid were made for MS analysis (MALDI-TOF). All MS spectra indicate the presence of full-length product without any truncated failure or deletion sequences.

Analysis of the products was performed by HPLC using a 4×23 mm YMC ODS-AQ (C18) column with the following conditions: 2.5-30% B/20 min gradient at 1.5 mL/min (0.1% TFA as modifier, A=H₂O, B=ACN). The crude was diluted 20 fold (10 uL into 190 uL H₂O), of which 30 uL was injected. All apgPNA oligomers synthesized were found to be of high purity (i.e., greater than 95%).

The crude apPNAs was purified by preparative HPLC, for example, with a 10×30 mm YMC ODS-AQ column. For the described system, a gradient of 2.5-35% B/25 min at 6 mL/min is used with the same solvent system as above. The pertinent fractions are analyzed and pooled by MS. The pooled fractions are quantified and lyophilized overnight. Table I below shows the recovery yields for a number of synthesized apgPNAs.

TABLE I
Recovery of apgPNA following purification.

| | | | | Crude | | Purified | | |
|---|---|---|---|---|---|---|---|---|
| ID | Length | MW | ε | OD-260 | nmol | OD-260 | nmol | Recovery |
| apPNA-1-12 | 12 | 3712.80 | 152.4 | 59.8 | 392.4 | 21.4 | 140.4 | 35.8% |
| apPNA-1-11 | 11 | 3407.50 | 140.7 | 64.1 | 455.6 | 22.5 | 159.9 | 35.1% |
| apPNA-1-10 | 10 | 3102.20 | 129.0 | 67.0 | 519.4 | 28.5 | 220.9 | 42.5% |
| apPNA-2-12 | 12 | 3744.80 | 148.4 | 69.8 | 470.4 | 25.2 | 169.8 | 36.1% |
| apPNA-2-11 | 11 | 3439.50 | 136.7 | 72.5 | 530.4 | 26.1 | 190.9 | 36.0% |
| apPNA-2-10 | 10 | 3134.20 | 125.0 | 48.2 | 385.6 | 28.2 | 225.6 | 58.5% |

All publications, patents, patent applications and other documents cited in this application are hereby incorporated by reference in their entireties for all purposes to the same extent as if each individual publication, patent, patent application or other document were individually indicated to be incorporated by reference for all purposes.

In the event that any definition or usage of a word or phrase used herein is in conflict with the definition and/or usage of that word or phrase in any other document, including any document incorporated herein by reference, the definition and/or usage of said word or phrase wherein shall always control.

While the present teachings are described in conjunction with various embodiments, it is not intended that the present teachings be limited to such embodiments. On the contrary, the present teachings encompass various alternatives, modifications, and equivalents, as will be appreciated by those of skill in the art.

1. A composition comprising:
(a) a first nucleobase polymer comprising a polypurine tract;
(b) a second nucleobase polymer comprising a polypyrimidine tract that is complementary to, and annealed to, the polypurine tract of the first nucleobase polymer, thereby forming a duplex comprising a duplex segment of poly(purine:polypyrimidine) base-pairs; and
(c) a third nucleobase polymer comprising a second polypurine tract that is complementary to the polypyrimidine tract of the duplex segment, wherein the third nucleobase polymer comprises a backbone of sufficient length such that each purine base of the second polypurine tract is hydrogen bonded to both purine and pyrimidine bases of a purine:pyrimidine base-pair of the duplex segment.

2. The composition of claim 1, wherein the backbone of the third nucleobase polymer is uncharged or comprises one or more positive charges.

3. The composition of claim 1, wherein the third nucleobase polymer comprises one or more linked positively charged groups.

4. The composition of claim 1, wherein the first and/or second nucleobase polymer comprises a sugar-phosphate backbone.

5. The composition of claim 4, wherein the attachment of the nucleobase to the sugar moiety of the sugar phosphate backbone of each polymer is, independently of the other, in the α or β conformation.

6. The composition of claim 1, further comprising an intercalator.

7. The composition of claim 6, wherein the intercalator is attached to a nucleobase of the third nucleobase polymer.

8. The composition of claim 6, wherein the intercalator is attached to the backbone of the third nucleobase polymer.

9. The composition of claim 6, wherein the intercalator is selected from anthracene, pyrene, 9-aminoacridine, daunomycin, and anthraquinone.

10. The composition of claim 1, wherein the first nucleobase polymer is annealed to the second nucleobase polymer by Watson-Crick base-pairing.

11. The composition of claim 1, wherein each of the purine bases of the second polypurine tract is selected from adenine, guanine, 2,6-diaminopurine, and isoguanine.

12. The composition of claim 1, wherein the third nucleobase polymer is a chimera of nucleobase polymers.

13. The composition of claim 1, wherein the first nucleobase polymer, second nucleobase polymer, and third nucleobase polymer are on a single strand.

14. The composition of claim 1, wherein the first nucleobase polymer and second nucleobase polymer are on a single strand.

15. The composition of claim 1, wherein the first and second nucleobase polymers are on separate strands.

16. The composition of claim 1, wherein the third nucleobase polymer is on a separate strand.

17. The composition comprising:
(a) a first nucleobase polymer comprising a first two polypurine tracts;
(b) a second nucleobase polymer comprising two polypyrimidine tracts complementary to, and annealed to, the first two polypurine tracts, thereby forming a duplex of a first and second duplex segments of poly(purine:polypyrimidine) base-pairs; and
(b) a third nucleobase polymer comprising a second two polypurine tracts complementary to the two polypyrimidine tracts of the first and second duplex segments, wherein the third nucleobase polymer comprises a backbone of sufficient length and a linker connecting the second two polypurine tracts to each other such that each purine base of the second two polypurine tracts is hydrogen bonded to both purine and pyrimidine bases of a purine:pyrimidine base-pair of the first or second duplex segment.

18. The composition comprising:
(a) a first nucleobase polymer comprising a first polypurine tract and first polypyrimidine tract;
(b) a second nucleobase polymer comprising a second polypyrimidine tract and second polypurine tract complementary to, and annealed to, the first polypurine tract and first polypyrimidine tract of the first nucleobase polymer, thereby forming a duplex comprising a first duplex segment of poly(purine:pyrimidine) base-pairs and a second duplex segment of poly(pyrimidine:purine) base-pairs; and
(c) a third nucleobase polymer comprising two polypurine tracts complementary to the first and second polypyrimidine tracts of the first and second duplex segments, wherein the third nucleobase polymer comprises a backbone of sufficient length and a linker connecting the two polypurine tracts to each other such that each purine base of the two polypurine tracts is hydrogen bonded to both purine and pyrimidine bases of a purine:pyrimidine base-pair of the first duplex segment or a pyrimidine:purine base-pair of the second duplex segment.

19. The composition of claim 17 or 18, wherein the backbone of the third nucleobase polymer is uncharged or comprises one or more positive charges.

20. The composition of claim 17 or 18, wherein the third nucleobase polymer comprises one or more linked positively charged groups.

21. The composition of claim 17 or 18, wherein the first and/or second nucleobase polymer comprises a sugar-phosphate backbone.

22. The composition of claim 21, wherein the attachment of the nucleobase to the sugar moiety of the sugar phosphate backbone of each strand is, independently of the other, in the α or β conformation.

23. The composition of claim 17 or 18, wherein the two polypurine tracts of the third nucleobase polymer are connected to the linker through one of either a carboxylic acid group or amine group on each respective tract.

24. The composition of claim 23, wherein the two polypurine tracts of the third nucleobase polymer are each connected to the linker through an amine group on the respective tracts.

25. The composition of claim 23, wherein the two polypurine tracts of the third nucleobase polymer are each connected to the linker through a carboxylic acid group on the respective tracts.

26. The composition of claim 17 or 18, further comprising an intercalator.

27. The composition of claim 26, wherein the intercalator is attached to the linker.

28. The composition of claim 26, wherein the intercalator is attached to a nucleobase of the third nucleobase polymer.

29. The composition of claim 26, wherein the intercalator is attached to the backbone of the third nucleobase polymer.

30. The composition of claim 26, wherein the intercalator is selected from anthracene, pyrene, 9-aminoacridine, daunomycin, and anthraquinone.

31. The composition of claim 17 or 18, wherein the first nucleobase polymer is annealed to the second nucleobase polymer by Watson-Crick base-pairing.

32. The composition of claim 17 or 18, wherein each of the purine bases of the second two polypurine tracts are selected from adenine, guanine, 2,6-diaminopurine, and isoguanine.

33. The composition of claim 17 or 18, wherein the third nucleobase polymer is a chimera of nucleobase polymers.

34. A composition according to structural formula (I): 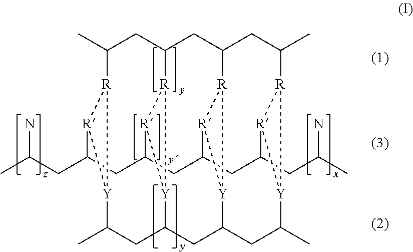
wherein:
(1) is a first nucleobase polymer;
(2) is a second nucleobase polymer; and
(3) is a third nucleobase polymer;
wherein:
each dashed line represents one or more hydrogen bonds between the nucleobases of the first, second and third nucleobase polymers;
each 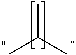
represents a backbone moiety of a subunit of each nucleobase polymer;
each N is, independently of the others, a nucleobase;
each R is, independently of the others, a purine nucleobase;
each Y is a pyrimidine nucleobase that is complementary to the R purine nucleobase to which it is hydrogen bonded;
each R′ is a purine nucleobase of the nucleobase containing subunit that is complementary to the Y pyrimidine nucleobase to which it is hydrogen bonded;
x is an integer ranging from 0 to 50;
y′ is an integer ranging from 2 to 30
y is an integer ranging from 2 to 30;
z is an integer ranging from 0 to 50.

35. The composition of claim 34, wherein y′=y.

36. The composition of claim 34, wherein
each N is, independently of the others, adenine, cytosine, guanine, thymine, 2-thiouracil, 2-thiothymine, pseudo-isocytosine, 2,6-diaminopurine or uracil;
each R is, independently of the others, adenine, guanine, isoguanine or 2,6-diaminopurine;
each Y is, independently of the others, cytosine, thymine, 2-thiouracil, 2-thiothymine, pseudo-isocytosine, or uracil; and
each R′ is, independently of the others, adenine, guanine, 2,6-diaminopurine, or isoguanine.

37. The composition of claim 34, wherein each 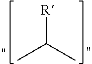
in the third strand represents a subunit according to structural formula (II): 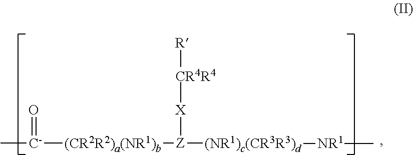
wherein:
each R¹ is independently H or lower alkyl;
each R² is independently H, lower alkyl, or alkylamine;
each R³ is independently H or lower alkyl, or alkylamine;
each R⁴ is independently H or lower alkyl;
a is 1, 2 or 3;
b is 0 or 1;
c is 0 or 1;
d is 1, 2, or 3;
Z is —CR¹— or N, wherein R¹ is defined as above;
X is —CR⁵R⁵—, —C(O)—, —C(S)—, or —NR¹—, wherein R¹ is defined as above, and each R⁵ is independently H or lower alkyl;
a+b+c+d=4; or
optionally wherein:
b+c=0, a is 1, d is 3, and
(i) R² and R⁴ together with Z and X;
(ii) R³ and R⁴ together with Z and X;
(iii) R² and R³ together with Z;
(iv) R² with Z; or
(v) R³ with Z
is a five or six membered cycloalkyl or heterocycloalkyl ring.

38. The composition of claim 37 wherein Z is N and X is —C(O)—.

39. The composition of claim 37 wherein Z is N, each R⁴ is H, and X is —CR⁵R⁵—, wherein each R⁵ is H.

40. The composition of claim 37 wherein “a” is 1 and “d” is 3.

41. The composition of claim 37 wherein “a” is 2 and “d” is 2.

42. The composition of claim 37, wherein each 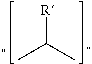
of the third nucleobase polymer represents a subunit according to structural formula (III):

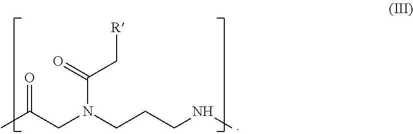

43. The composition of claim 37, wherein each 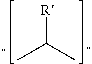
in the third nucleobase polymer represents a subunit according to structural formula (IV):

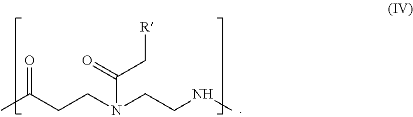

44. The composition of claim 37, wherein each 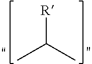
in the third nucleobase polymer represents a subunit according to structural formula (V):

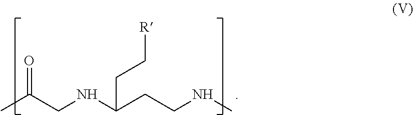

45. The composition of claim 37, wherein in which each 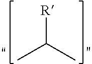
in the third nucleobase polymer is a subunit according to the following structures:

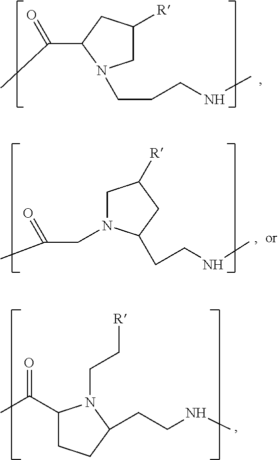

46. The composition of claim 1, wherein the backbone of the first and/or second nucleobase polymer comprises a 2′-deoxyribophosphate.

47. The composition of claim 1, wherein the backbone of the first and/or second strand comprises a 2′-ribophosphate.

48. The composition of claim 37, wherein the amino terminus of the third nucleobase polymer is oriented towards the 5-prime terminus of the first strand.

49. The composition of claim 37, wherein the carboxy terminus of the third strand is oriented towards the 5-prime terminus of the first strand.

50. The composition of claim 37, wherein the third nucleobase polymer comprises a label

51. The composition of claim 50, wherein the label comprises a chromophore.

52. The composition of claim 51, wherein the chromophore comprises a fluorophore.

53. The composition of claim 50, wherein the chromophore comprises an acceptor or donor chromophore, and the first and/or second nucleobase polymer further comprises a corresponding donor or acceptor chromophore to form a donor-acceptor chromophore pair with the chromophore of the nucleobase polymer, wherein the donor and acceptor chromophore pair is suitably positioned to permit energy transfer between the donor and acceptor chromophores.

54. The composition of claim 53, wherein the chromophore of the third nucleobase polymer comprises a donor or acceptor.

55. The composition of claim 54, wherein the chromophore is a FRET acceptor.

56. The composition of claim 54, wherein the chromophore is a FRET donor.

57. The composition of claim 53 wherein the chromophore of the third nucleobase polymer is one of either a fluorescence quencher or a fluorophore and the chromophore of the first and/or second nucleobase polymer is the other one of either a fluorescence quencher or a fluorophore.

58. The composition of claim 50, wherein the third strand comprises both a donor and acceptor chromophore.

59. The composition of claim 34 or 58, wherein the third nucleobase polymer comprises an intercalator.

60. The composition of claim 59 wherein the intercalator is attached to the backbone.

61. The composition of claim 59, wherein the intercalator is attached to the third nucleobase polymer through a linker.

62. The composition of claim 59, wherein the intercalator is attached to at least one of the R′ adjacent to the left or right N.

63. The composition of claim 59 wherein the intercalator is attached to the left R′ residue adjacent to the left N and to the right R′ adjacent to the right N.

64. The composition of claim 59, wherein the intercalator is selected from anthracene, pyrene, 9-aminoacridine, daunomycin, and anthraquinone.

65. The composition of claim 34, wherein the first and/or second nucleobase polymer is part of a polynucleotide.

66. The composition of claim 65, wherein the polynucleotide is genomic DNA or an amplified polynucleotide.

67. The composition of claim 65, wherein the polynucleotide is a chromosome.

68. The composition of claim 34, wherein the first, second or third nucleobase polymer is attached to a substrate.

69. The composition of claim 68, wherein the substrate is selected from glass, plastic, metal, and silicon.

70. A method comprising:
(a) forming the composition of claim 1, wherein the first and second nucleobase polymers comprise a double-stranded target polynucleotide; and
(b) detecting the composition.

71. The method of claim 70, wherein the method comprises amplifying a nucleic acid of in erect to generate the double-stranded target polynucleotide.

72. The method of claim 71, wherein the third nucleobase polymer is added before, during, or after the amplification reaction.

73. The method of claim 71, wherein the amplification reaction is a polymerase chain reaction.

74. The method of claim 70, wherein the presence, absence, and/or quantity of the composition is detected by electrophoresis.

75. The method of claim 70, wherein the presence, absence, and/or quantity of the composition is detected by fluorescence.

76. The method of claim 70, wherein the presence, absence, and/or quantity of the composition is detected by fluorescence resonance energy transfer (FRET).

77. The method of claim 70, wherein the presence, absence, and/or quantity of the composition is detected by fluorescence quenching.

78. The method of claim 77, wherein fluorescence quenching occurs by FRET quenching

79. The method of claim 77, wherein fluorescence quenching occurs by non-FRET quenching.

80. The method of claim 77, wherein fluorescence quenching occurs by a combination of FRET and non-FRET quenching.
